(19) United States
(12) Patent Application Publication
(10) Pub. No.: US 2026/0018306 A1
(43) Pub. Date: Jan. 15, 2026

(54) APPARATUS AND METHOD FOR GENERATING A PREOPERATIVE DATA STRUCTURE USING A PRE-OPERATIVE PANEL

(71) Applicant: Anumana, Inc., Cambridge, MA (US)

(72) Inventors: Samir Awasthi, Boston, MA (US); Shahir Asfahan, Bengaluru (IN); Shashi Kant, Bengaluru (IN); Rakesh Barve, Bengaluru (IN); Lars Hegstrom, Cambridge, MA (US)

(73) Assignee: Anumana, Inc., Cambridge, MA (US)

(21) Appl. No.: 18/773,195

(22) Filed: Jul. 15, 2024

(51) Int. Cl. G16H 70/20; G16H 10/60

(52) U.S. Cl. CPC G16H 70/20; G16H 10/60

(57) ABSTRACT

An apparatus and method for generating a preoperative data structure using a pre-operative panel are disclosed. The apparatus includes a memory containing instructions configuring at least a processor to receive subject data including ECG data, generate a plurality of panel outputs as a function of the subject data using a pre-operative panel machine-learning module including a plurality of panel machine-learning models, wherein each of the plurality of panel machine-learning models is configured to generate one panel output for one panel focus, wherein generating the plurality of panel outputs includes generating a plurality of sets of panel training data, training each of the plurality of panel machine-learning models using each of the plurality of sets of panel training data and generating the plurality of panel outputs using the plurality of trained panel machine-learning models and generate a pre-operative data structure as a function of the plurality of panel outputs.

### FIELD OF THE INVENTION

The present invention generally relates to the field of medical system. In particular, the present invention is directed to an apparatus and method for generating a preoperative data structure using a pre-operative panel.

### BACKGROUND

Surgical procedures, both elective and emergency, necessitate comprehensive preoperative evaluations to ensure patient safety and optimize outcomes. Traditional preoperative assessments typically include a range of individual tests. However, the current approach to preoperative testing can be time-consuming and inefficient. The proposed invention focuses on a novel preoperative panel system designed to address the limitations of current preoperative evaluation methods.

### SUMMARY OF THE DISCLOSURE

In an aspect, an apparatus for generating a preoperative data structure using a pre-operative panel is disclosed. The apparatus includes at least a processor and a memory communicatively connected to the at least a processor, wherein the memory contains instructions configuring the at least a processor to receive subject data, wherein the subject data includes electrocardiogram (ECG) data, generate a plurality of panel outputs as a function of the subject data using a pre-operative panel machine-learning module, wherein the pre-operative panel machine-learning module includes a plurality of panel machine-learning models, wherein each of the plurality of panel machine-learning models is configured to generate one panel output for one panel focus as a function of the subject data, wherein generating the plurality of panel outputs includes generating a plurality of sets of panel training data, wherein the plurality of sets of panel training data includes correlations between exemplary subject data, exemplary panel focuses and exemplary panel outputs, training each of the plurality of panel machine-learning models using each of the plurality of sets of panel training data and generating the plurality of panel outputs using the plurality of trained panel machine-learning models and generate a pre-operative data structure as a function of the plurality of panel outputs.

In another aspect, a method for generating a preoperative data structure using a pre-operative panel is disclosed. The method includes receiving, using at least a processor, subject data, wherein the subject data includes electrocardiogram (ECG) data, generating, using the at least a processor, a plurality of panel outputs as a function of the subject data using a pre-operative panel machine-learning module, wherein the pre-operative panel machine-learning module includes a plurality of panel machine-learning models, wherein each of the plurality of panel machine-learning models is configured to generate one panel output for one panel focus as a function of the subject data, wherein generating the plurality of panel outputs includes generating a plurality of sets of panel training data, wherein the plurality of sets of panel training data includes correlations between exemplary subject data, exemplary panel focuses and exemplary panel outputs, training each of the plurality of panel machine-learning models using each of the plurality of sets of panel training data and generating the plurality of panel outputs using the plurality of trained panel machine-learning models and generating, using the at least a processor, a pre-operative data structure as a function of the plurality of panel outputs.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a block diagram of an exemplary apparatus for generating a preoperative data structure using a pre-operative panel;

FIG. 2 illustrates an exemplary panel data structure displayed on a user device;

FIG. 3 illustrates a block diagram of an exemplary subject database;

FIG. 4 illustrates a systemic diagram of an exemplary chatbot system;

FIG. 5 illustrates a block diagram of exemplary embodiment of a machine learning module;

FIG. 6 illustrates a diagram of an exemplary neural network;

FIG. 7 illustrates a block diagram of an exemplary node in a neural network;

FIG. 8 illustrates a block diagram of an exemplary pre-operative panel machine-learning module;

FIG. 9 illustrates a flow diagram of an exemplary method for generating a preoperative data structure using a pre-operative panel; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatuses and methods for generating a preoperative data structure using a pre-operative panel. The apparatus includes at least a processor and a memory communicatively connected to the at least a processor, wherein the memory contains instructions configuring the at least a processor to receive subject data, wherein the subject data includes ECG data, generate a plurality of panel outputs as a function of the subject data using a pre-operative panel machine-learning module, wherein the pre-operative panel machine-learning module includes a plurality of panel machine-learning models, wherein each of the plurality of panel machine-learning models is configured to generate one panel output for one panel focus as a function of the subject data, wherein generating the plurality of panel outputs includes generating a plurality of sets of panel training data, wherein the plurality of sets of panel training data includes correlations between exemplary subject data, exemplary panel focuses and exemplary panel outputs, training each of the plurality of panel machine-learning models using each of the plurality of sets of panel training data and generating the plurality of panel outputs using the plurality of trained panel machine-learning models and generate a pre-operative data structure as a function of the plurality of panel outputs. Exemplary embodiments illustrating aspects of the present disclosure are described below in the context of several specific examples.

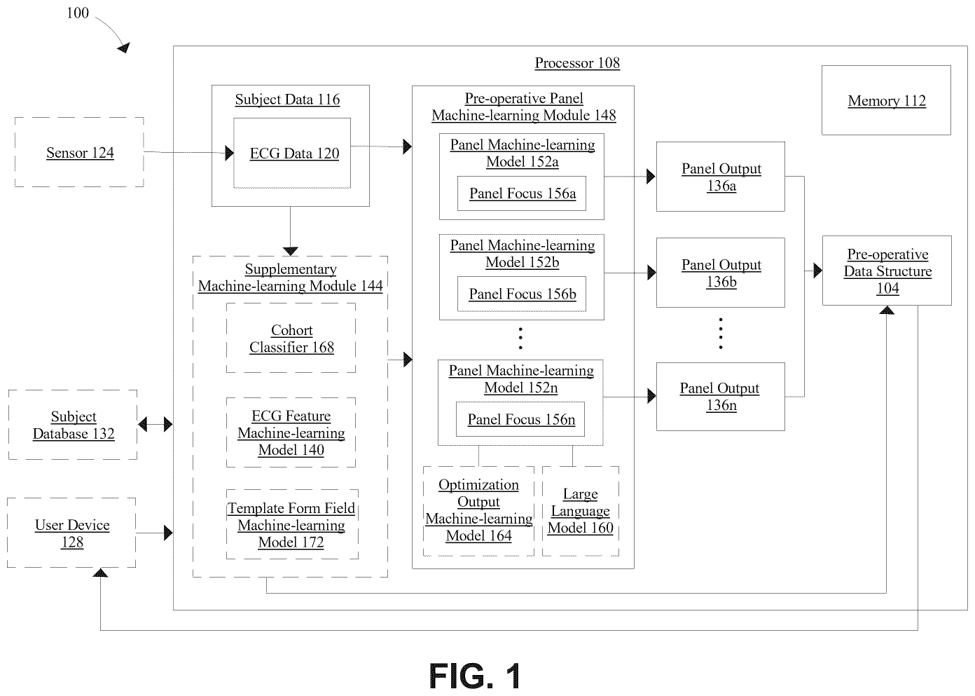

Referring now to FIG. 1, an exemplary embodiment of an apparatus 100 for generating a pre-operative data structure 104 using a pre-operative panel is illustrated. Apparatus 100 includes at least a processor 108. Processor 108 may include, without limitation, any processor described in this disclosure. Processor 108 may be included in a computing device. Processor 108 may include any computing device as described in this disclosure, including without limitation a microcontroller, microprocessor, digital signal processor (DSP) and/or system on a chip (SoC) as described in this disclosure. Processor 108 may include, be included in, and/or communicate with a mobile device such as a mobile telephone or smartphone. Processor 108 may include a single computing device operating independently, or may include two or more computing device operating in concert, in parallel, sequentially or the like; two or more computing devices may be included together in a single computing device or in two or more computing devices. Processor 108 may interface or communicate with one or more additional devices as described below in further detail via a network interface device. Network interface device may be utilized for connecting processor 108 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 108 may include but is not limited to, for example, a computing device or cluster of computing devices in a first location and a second computing device or cluster of computing devices in a second location. Processor 108 may include one or more computing devices dedicated to data storage, security, distribution of traffic for load balancing, and the like. Processor 108 may distribute one or more computing tasks as described below across a plurality of computing devices of computing device, which may operate in parallel, in series, redundantly, or in any other manner used for distribution of tasks or memory between computing devices. Processor 108 may be implemented, as a non-limiting example, using a “shared nothing” architecture.

With continued reference to FIG. 1, processor 108 may be designed and/or configured to perform any method, method step, or sequence of method steps in any embodiment described in this disclosure, in any order and with any degree of repetition. For instance, processor 108 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 108 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory 112 communicatively connected to processor 108. For the purposes of this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 112 contains instructions configuring processor 108 to receive subject data 116. For the purposes of this disclosure, “subject data” is information related to a subject's health, medical history, or treatment. For the purposes of this disclosure, a “subject” is an individual who receives medical care, service or treatment. For example, and without limitation, subject may include a patient. As a non-limiting example, subject data 116 may include personal information. For example, and without limitation, personal information may include name, age, gender, date of birth, weight, address, contact information, or the like. As another non-limiting example, subject data 116 may include medical history, medications, allergies, diagnoses, lab results, pre-existing conditions, progress notes, alcohol consumption, smoking habit, exercise habit, vaccination history, vital signs, family medical history, or the like. As another non-limiting example, subject data 116 may include electromyogram (EMG) data, Electrooculogram (EOG) data, X-Ray imaging data, MRI (magnetic resonance imaging) data, CT (Computed Tomography) data, ultrasound data, Electroencephalograms (EEG) data, Echocardiograms (Echo) data, or the like.

With continued reference to FIG. 1, subject data 116 includes ECG data 120. For the purposes of this disclosure, “ECG data” is information related to electrocardiogram related to a subject. In one or more embodiments, ECG data 120 may include a matrix having a plurality of electrocardiogram signals and/or associated time variables. A “matrix” for the purposes of this disclosure is an array of numbers or characters arranged in rows or columns which are used to represent an object or properties of the object. For example, and without limitation, a matrix may be used to describe linear equations, differential equations, in a two-dimensional format. In another non limiting example, a matrix may be used to create graphs based on data points, generate statistical models and the like. In one or more embodiments, matrix may include a plurality of electrocardiogram signals associated with a plurality of time variables. As used in the current disclosure, a “electrocardiogram signal” is a signal representative of electrical activity of heart. The ECG signal may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves may provide information about the duration and regularity of various phases of the cardiac cycle. The ECG signal may help diagnose various heart conditions, such as arrhythmias, myocardial infarction (heart attack), conduction abnormalities, and electrolyte imbalances. In one or more embodiments, ECG signals may be received by one or more electrodes connected to the skin of an individual. In one or more embodiments, ECG signals may represent depolarization and repolarization occurring in the heart. In one or more embodiments, ECG signals may be captured periodically. For example, and without limitation, every second, every millisecond and the like.

With continued reference to FIG. 1, in one or more embodiments, each ECG signal may contain an associated time variable. “Time variable” for the purposes of this disclosure is information indicating the time at which a particular ECG signal was received. For example, and without limitation, time variable may include, 5 ms, 10 ms, 15 ms, and the like. In one or more embodiments, each ECG signal may contain a time variable. In one or more embodiments, time variable may increase in given increments, such as for example, in increments of 5 ms, wherein a first time variable may include 5 ms and a second time variable may include 10 ms. In one or more embodiments, a combination of a plurality of ECG signals and correlated time variable may be used to generate a graph illustrating the heart functions of an individual. In one or more embodiments, matrix may include a plurality of ECG signals and correlated time variable during a given time frame such as, for example, over the span of a second, a minute, an hour, and the like. In one or more embodiments, ECG signals may be captured as voltages, such as millivolts or microvolts.

With continued reference to FIG. 1, in some embodiments, processor 108 may receive subject data 116 and/or ECG data 120 from at least a sensor 124. For the purposes of this disclosure, a “sensor” is a device that produces an output signal for the purpose of sensing a physical phenomenon. As a non-limiting example, sensor 124 may include an ECG sensor. For the purposes of this disclosure, an “ECG sensor” is a device that detects and records the electrical signals produced by the heart during each heartbeat. For the purposes of this disclosure, an “electrode” is a conductive material or element that facilitates the transmission and reception of electrical signals associated with ultrasound waves. In a non-limiting example, electrode may detect and record electrical activity; for instance, but not limited to, the heart's electrical signals (e.g., ECG signals). In some embodiments, ECG sensor may generate a lead system and collect electrical signals using the leads. For the purposes of this disclosure, a “lead system” is the specific electrode placements on the body and the corresponding electrical views of the heart's activity they provide. As a non-limiting example, ECG signals may be collected using 1, 3, 6, and/or 12 lead systems. In some embodiments, a single-lead ECG may correspond to one of the leads of a 12-lead ECG. For example, and without limitation, one of the leads of the 12-lead ECG to which the single-lead corresponds may be Lead 1, Lead 2, Lead 3, AvF, AvL, AvR, or V1-V6. As another non-limiting example, sensor 124 may include EEG sensor, pulse oximeter, blood pressure monitor, glucose sensor, temperature sensor, wearable fitness tracker, or the like.

With continued reference to FIG. 1, in some embodiments, processor 108 may receive subject data 116 and/or ECG data 120 from a user device. For the purposes of this disclosure, a “user device” is any device a user uses to input data. As a non-limiting example, user device 128 may include a laptop, desktop, tablet, mobile phone, smart phone, smart watch, kiosk, smart headset, or things of the like. For the purposes of this disclosure, a “user” is any individual or entity that uses an apparatus 100. As a non-limiting example, user may include a health care provider, hospital, health organization, or the like. In some embodiments, user device 128 may include an interface configured to receive inputs from user. In some embodiments, user may manually input any data into apparatus 100 using user device 128. In some embodiments, user may have a capability to process, store or transmit any information independently.

With continued reference to FIG. 1, in some embodiments, subject data 116 and ECG data 120 may be stored in a subject database 132. In some embodiments, processor 108 may retrieve subject data 116 and ECG data 120 from subject database 132. In some embodiments, apparatus 100 may include a subject database 132. As used in this disclosure, “subject database” is a data structure configured to store data associated with a subject. In one or more embodiments, subject database 132 may include inputted or calculated information and datum related to a subject. In some embodiments, a datum history may be stored in subject database 132. As a non-limiting example, the datum history may include real-time and/or previous inputted data related to a subject. As a non-limiting example, subject database 132 May include instructions from a user, who may be an expert user, a past user in embodiments disclosed herein, or the like, where the instructions may include examples of the data related to a subject.

With continued reference to FIG. 1, processor 108 may receive subject data 116 and/or ECG data 120 from externally supplied databases. As a non-limiting example, ECG data 120 and/or subject data 116 may be retrieved from subject records. For the purposes of this disclosure, a “subject record” is comprehensive documentation contained medical information about an individual, including medical history, diagnoses, treatments, medications, laboratory results, compiled for healthcare purposes. As a non-limiting example, subject record may include electric health records (EHR). In some embodiments, the receipt of subject records may include communicating with a database (e.g., subject database 132) or databases responsible for hosting subject medical record information. In some embodiments, subject records may be included in a virtual private network, or a virtual private cloud locally stored on medical facilities or offsite. In some embodiments, subject records may be received over a communications protocol. For the purposes of this disclosure, “communications protocol” is a set of rules describing how to transmit, exchange or receive data across a network. It should be noted that the protocol used in communicating with a database may be standardized or unstandardized and be text-based, binary, or some other base.

With continued reference to FIG. 1, in some embodiments, processor 108 may be communicatively connected with subject database 132. For example, and without limitation, in some cases, subject database 132 may be local to processor 108. In another example, and without limitation, subject database 132 may be remote to processor 108 and communicative with processor 108 by way of one or more networks. The network may include, but is not limited to, a cloud network, a mesh network, and the like. By way of example, a “cloud-based” system can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 108 connect directly, dynamically, and non-hierarchically to as many other computing devices as possible. A “network topology” as used in this disclosure is an arrangement of elements of a communication network. The network may use an immutable sequential listing to securely store subject database 132. An “immutable sequential listing,” as used in this disclosure, is a data structure that places data entries in a fixed sequential arrangement, such as a temporal sequence of entries and/or blocks thereof, where the sequential arrangement, once established, cannot be altered or reordered. An immutable sequential listing may be, include and/or implement an immutable ledger, where data entries that have been posted to the immutable sequential listing cannot be altered.

With continued reference to FIG. 1, in some embodiments, subject database 132 may include keywords. As used in this disclosure, a “keyword” is an element of word or syntax used to identify and/or match elements to each other. For example, without limitation, the keyword may include a name of a subject in the instance that a user is looking for information related to the specific subject. For example, without limitation, the keyword may include a name of a disease or health condition in the instance that a user is looking for information related to the specific disease or health condition.

With continued reference to FIG. 1, in some embodiments, subject database 132 may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

With continued reference to FIG. 1, in some embodiments, processor 108 may be configured to extract or determine at least an ECG feature as a function of ECG data 120. For the purposes of this disclosure, an “ECG feature” is a specific characteristic or element observed in ECG data. In some embodiments, ECG feature may provide valuable information about the heart's electrical activity and overall function. As a non-limiting example, ECG feature may include time domain features, frequency domain features, morphological features, heart rate dynamics, nonlinear dynamics features and/or others. In some embodiments, ECG feature may be stored in subject database 132. In some embodiments, ECG feature may be retrieved from subject database 132. In some embodiments, user may manually input ECG feature.

With continued reference to FIG. 1, as a non-limiting example, ECG features may include morphological features which are characteristics of ECG waveforms such as amplitudes, duration, and shape of P, QRS and T waves, frequency domain features, which may include power spectral density of ECG signals across different frequency bands, heart rate variability metrics which may include measures of variation in heart rate intervals, such as SDNN (standard deviation of normal-to-normal intervals and RMSSD (root mean square of successive differences), or the like. In some embodiments, morphological features may include amplitude and duration of ECG waves which includes features such as P-wave amplitude and duration, QRS complex amplitude and duration, T-wave amplitude and duration etc., capturing characteristics of cardiac depolarization and repolarization. Heart rate dynamics may include heart rate turbulence (HRT) which quantifies the fluctuations in heart rate following premature ventricular contractions, providing insights to cardio autonomic regulation and risk of arrhythmias. Heart rate dynamics may further include heart rate variability patterns which includes features such as SDNN (standard deviation of normal-to-normal intervals) and RMSSD (root mean square of successive differences), reflecting overall variability and short-term fluctuations in heart rate, respectively. Nonlinear dynamics features may include Poincaré Plot analysis which characterizes the scatterplot of successive RR intervals, providing information about heart rate variability patterns and autonomic nervous system modulation. Nonlinear dynamics may further include approximate entropy which measures the irregularity or complexity of the ECG waveform, indicating the degree of cardiac dynamics and potential arrhythmogenicity.

With continued reference to FIG. 1, in some embodiments, time domain features may include heart rate variability (HRV) which measures the variation in time intervals between successive heartbeats, reflecting autonomic nervous system activity and cardiovascular health. Time domain features may further include QRS complex duration which represents the duration of the QRS complex on the ECG waveform, indicating ventricular depolarization time and potential conduction abnormalities. Frequency domain features may include power spectral density which quantifies the distribution of power across different frequency bands (e.g. very low frequency, low frequency, high frequency etc.) derived from the ECG signal, providing insights into automatic modulation of heart rate. Frequency domain may further include spectral entropy which measures the complexity or irregularity of the ECG waveform in the frequency domain, reflecting the balance between sympathetic and parasympathetic nervous system activity.

With continued reference to FIG. 1, in some embodiments, each of ECG features may contribute to constructing a multi-dimensional feature vector representing ECG signals. For example, a feature vector might look like: Feature Vector: [HRV, QRS Duration, Power in Very Low Frequency Band, Power in Low Frequency Band, Power in High Frequency Band, Spectral Entropy, P-wave Amplitude, P-Wave Duration, QRS Amplitude, QRS Duration, T-Wave Amplitude, T-Wave Duration, HRT Parameters, Nonlinear Dynamics Features]. Each component of this feature vector captures different aspects of cardiac activity derived from ECG signals, enabling machine learning algorithms to analyze and classify cardiac function, detect abnormalities, and assess surgical risk in pre-operative assessments, which results in generating panel outputs 136a-n.

With continued reference to FIG. 1, in some embodiments, processor 108 may determine ECG feature using an ECG feature machine-learning model 140. In some embodiments, supplementary machine-learning module 144 may include ECG feature machine-learning model 140. The supplementary machine-learning module 144 is further described below. In some embodiments, processor 108 may be configured to generate ECG feature training data. In a non-limiting example, ECG feature training data may include correlations between exemplary ECG data and exemplary ECG features. In some embodiments, ECG feature training data may be stored in subject database 132. In some embodiments, ECG feature training data may be received from one or more users, subject database 132, external computing devices, and/or previous iterations of processing. As a non-limiting example, ECG feature training data may include instructions from a user, who may be an expert user, a past user in embodiments disclosed herein, or the like, which may be stored in memory and/or stored in subject database 132, where the instructions may include labeling of training examples. In some embodiments, ECG feature training data may be updated iteratively using a feedback loop. As a non-limiting example, processor 108 may update ECG feature training data iteratively through a feedback loop as a function of subject data 116, ECG data 120, output of any machine-learning models described in this disclosure, or the like. In some embodiments, processor 108 may be configured to generate ECG feature machine-learning model 140 of supplementary machine-learning module 144. In a non-limiting example, generating ECG feature machine-learning model 140 may include training, retraining, or fine-tuning ECG feature machine-learning model 140 using ECG feature training data or updated ECG feature training data. In some embodiments, processor 108 may be configured to determine ECG feature using ECG feature machine-learning model 140 (i.e. trained or updated ECG feature machine-learning model 140).

With continued reference to FIG. 1, in some embodiments, processor 108 may analyze ECG data 120 using a machine vision system to determine ECG feature. For the purposes of this disclosure, a “machine vision system” is a type of technology that enables a computing device to inspect, evaluate and identify still or moving images. In a non-limiting example, ECG data may include an image of a matrix of ECG signals and processor 108 may analyze the image using machine vision system to determine ECG feature. For example, and without limitation, a machine vision system may be used for world modeling or registration of objects within a space. In some cases, registration may include image processing, such as without limitation object recognition, feature detection, edge/corner detection, and the like. Non-limiting example of feature detection may include scale invariant feature transform (SIFT), Canny edge detection, Shi Tomasi corner detection, and the like. In some cases, a machine vision process may operate image classification and segmentation models, such as without limitation by way of machine vision resource (e.g., OpenMV or TensorFlow Lite). A machine vision process may detect motion, for example by way of frame differencing algorithms. A machine vision process may detect markers, for example blob detection, object detection, face detection, and the like. In some cases, a machine vision process may perform eye tracking (i.e., gaze estimation). In some cases, a machine vision process may perform person detection, for example by way of a trained machine learning model. In some cases, a machine vision process may perform motion detection (e.g., camera motion and/or object motion), for example by way of optical flow detection. In some cases, machine vision process may perform code (e.g., barcode) detection and decoding. In some cases, a machine vision process may additionally perform image capture and/or video recording.

With continued reference to FIG. 1, in some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ø may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level.

With continued reference to FIG. 1, alternatively or additionally, determining ECG feature may include analyzing ECG data 120 using an image classifier; the image classifier May be trained using a plurality of images representing ECG features. As a non-limiting example, image classifier may be trained with image training data that correlates exemplary ECG data to exemplary ECG features. Alternatively, determination or extraction of ECG features may be performed without using machine vision and/or classification; for instance, extracting ECG features may further include receiving, from a user, an identification of ECG feature in an image of ECG data 120. In some embodiments, machine vision system may use image classifier. In some embodiments, supplementary machine-learning module 144 may include image classifier.

With continued reference to FIG. 1, additional disclosure related to ECG feature may be found in U.S. Non-provisional patent application Ser. No. 18/230,043, filed on Aug. 3, 2023, entitled “APPARATUS AND A METHOD FOR GENERATING A DIAGNOSTIC LABEL,” having an attorney docket number of 1518-102USU1, and U.S. Non-provisional patent application Ser. No. 18/652,921, filed on May 2, 2024, entitled “APPARATUS AND METHOD FOR CLASSIFYING A USER TO A COHORT OF RETROSPECTIVE USERS,” having an attorney docket number of 1518-144USU1, the entirety of each of which is incorporated herein as reference.

With continued reference to FIG. 1, memory 112 contains instructions configuring processor 108 to generate a plurality of panel outputs 136a-n. For the purposes of this disclosure, a “panel output” is one or more results involving a evaluation of subject data to assess various aspects of a subject's health. As a non-limiting example, panel outputs 136a-n may include text, image, icon, or the like. For example, and without limitation, panel outputs 136a-n may include Boolean value; Yes/No, True/False, or the like. For example, and without limitation, panel outputs 136a-n may include a numerical value; a percentage of risk of surgical procedure, a percentage of medical evaluation result, possibility of having a certain disease or medical condition, or the like. For example, and without limitation, panel outputs 136a-n may include textual output; an answer to a question, description of a medical condition, analysis of ECG data 120, sources related to a medical condition, or the like. In some embodiments, panel outputs 136a-n may be stored in subject database 132. In some embodiments, panel outputs 136a-n may be retrieved from subject database 132. In some embodiments, a user may manually input panel outputs 136a-n.

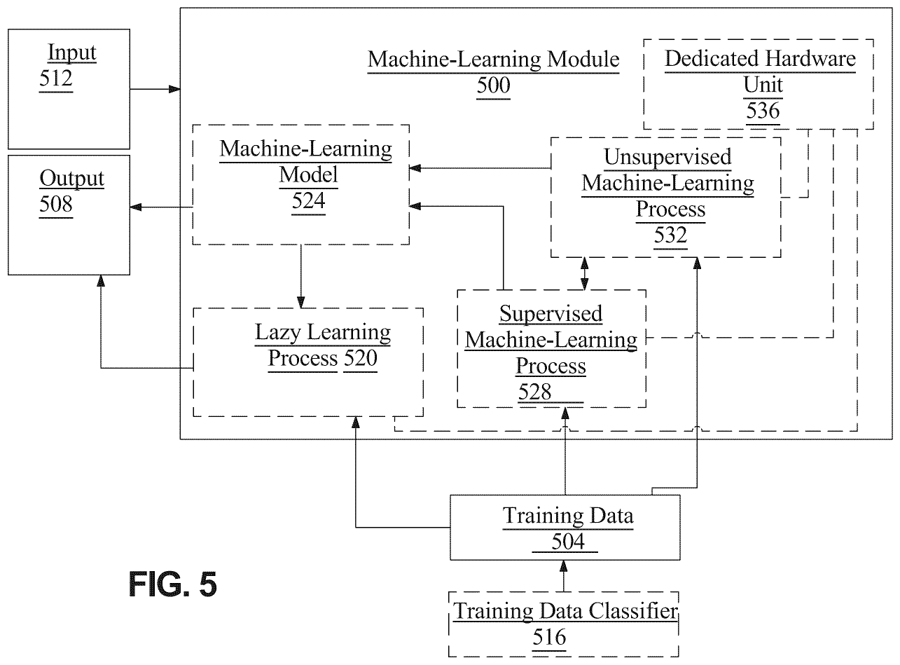

With continued reference to FIG. 1, processor 108 is configured to generate panel outputs 136a-n as a function of subject data 116 using a pre-operative panel machine-learning module 148. For the purposes of this disclosure, a “pre-operative panel machine-learning module” is a component or package that provides functionalities for building, training, and deploying machine-learning models for making pre-operative decision. For the purposes of this disclosure, “pre-operative” refers to before a surgical procedure takes place. For the purposes of this disclosure, a “pre-operative panel” is a set of medical evaluations conducted before a surgical procedure to assess a subject's overall health and readiness for surgery. Pre-operative panel machine-learning module 148 disclosed herein may be consistent with machine-learning module described with respect to FIG. 5. Pre-operative panel machine-learning module 148 includes a plurality of panel machine-learning models 152a-n. For the purposes of this disclosure, a “panel machine-learning model” is a mathematical model, neural net, or program generated by a machine learning algorithm that generates a panel output correlated to inputted data. As a non-limiting example, plurality of panel machine-learning models 152a-n may include a first panel machine-learning model 152a, second panel machine-learning model 152b, n number of panel machine-learning model 152n, or the like.

With continued reference to FIG. 1, each of plurality of panel machine-learning models 152a-n is configured to generate one panel output 136a-n for one panel focus 156a-n as a function of subject data 116. For the purposes of this disclosure, a “panel focus” is primary subject matter that at least a test of a panel will concentrate on to make a pre-operative decision related to a medical or health condition. As a non-limiting example, panel focus 156a-n may include any medical conditions thereof. For example, and without limitation, panel focus 156a-n may include ejection fraction, coronary heart disease, hypertensive heart disease, pulmonary hypertension, atrial fibrillation, cardio myopathy, diastolic dysfunction, cardiac amyloidosis, left ventricular filling pressure (LVFP), cardiac pulmonary arrest, or the like. For example, and without limitation, panel focus 156a-n may include complete blood count, coagulation studies, allergies, abnormal breathing, or the like. For example, and without limitation, panel focus 156a-n may include a possibility of death before or after a surgery, necessity to get a surgery, alternative treatments, risks or complications of a surgery, type of anesthesia, cost of surgery, recovery time, or the like. In a non-limiting example, panel machine-learning model 152a-n that includes panel focus 156a-n related to cardiac pulmonary arrest may output a percentage or Boolean value reflecting a possibility of needing cardiopulmonary resuscitation as panel output 136a-n.

With continued reference to FIG. 1, for the purposes of this disclosure, “ejection fraction” is a measurement used to assess how well the heart is pumping blood. For the purposes of this disclosure, “coronary heart disease” is a condition characterized by the narrowing or blockage of the coronary arteries. For the purposes of this disclosure, “pulmonary hypertension” is a condition characterized by elevated blood pressure in the arteries of the lungs. For the purposes of this disclosure, “atrial fibrillation” is a type of arrhythmia, or irregular heartbeat, characterized by rapid and disorganized electrical signals in the atria, the upper chambers of the heart. For the purposes of this disclosure, “diastolic dysfunction” is the impaired ability of the heart's ventricles to relax and fill with blood during the diastolic phase of the cardiac cycle. For the purposes of this disclosure, “cardiac amyloidosis” is a form of amyloidosis where amyloid proteins deposit in the heart tissue. For the purposes of this disclosure, “left ventricular filling pressure” is the pressure in the left ventricle during diastole, when the heart is filling with blood. For the purposes of this disclosure, “cardiac pulmonary arrest” is an event where the heart stops pumping blood effectively, leading to the cessation of blood flow to the brain and other vital organs.

With continued reference to FIG. 1, in some embodiments, each plurality of panel machine-learning models 152a-n may include one panel focus 156a-n that is different compared to others. As a non-limiting example, first panel machine-learning model 152a may include a first panel focus 156a related to ejection fraction, second panel machine-learning model 152b may include a second panel focus 156b related to coronary heart disease, and the like. In a non-limiting example, plurality of panel machine-learning models 152a-n may include a first panel machine-learning model 152a including a first panel focus 156a related to coronary heart disease, wherein the first panel machine-learning model 152a may be configured to generate a first panel output 136a related to the coronary heart disease as a function of subject data 116. Continuing non-limiting example, plurality of panel machine-learning models 152a-n may include a second panel machine-learning model 152b including a second panel focus 156b related to pulmonary hypertension, wherein the second panel machine-learning model 152b may be configured to generate a second panel output 136b related to the pulmonary hypertension as a function of subject data 116. Continuing non-limiting example, plurality of panel machine-learning models 152a-n may include a third panel machine-learning model 152c including a third panel focus 156c related to atrial fibrillation, wherein the third panel machine-learning model 152c may be configured to generate a third panel output 136c related to the atrial fibrillation as a function of subject data 116. Continuing non-limiting example, plurality of panel machine-learning models 152a-n may include a fourth panel machine-learning model 152d including a fourth panel focus 156d related to ejection fraction, wherein the fourth panel machine-learning model 152d may be configured to generate a fourth panel output 136d related to the ejection fraction as a function of subject data 116. Panel focuses 156a-n disclosed herein are merely examples and persons skilled in the art, upon reviewing the entirety of this disclosure, may appreciate various panel focuses 156a-n that can be used in apparatus 100.

With continued reference to FIG. 1, processor 108 is configured to generate a plurality of sets of panel training data. For the purposes of this disclosure, “panel training data” is data containing correlations that a machine-learning process may use to model relationships to generate a panel output. Plurality of sets of panel training data includes correlations between exemplary subject data, exemplary panel focuses and exemplary panel outputs. In a non-limiting example, plurality of sets of panel training data may include correlations between exemplary subject data, exemplary ECG features, exemplary panel focuses and exemplary panel outputs For example, and without limitation, first set of panel training data may include correlations between exemplary subject data, exemplary first panel focus and exemplary panel outputs. For example, and without limitation, second set of panel training data may include correlations between exemplary subject data, exemplary second panel focus and exemplary panel outputs. In some embodiments, panel training data may be stored in subject database 132. In some embodiments, panel training data may be received from one or more users, subject database 132, external computing devices, and/or previous iterations of processing. As a non-limiting example, panel training data may include instructions from a user, who may be an expert user, a past user in embodiments disclosed herein, or the like, which may be stored in memory and/or stored in subject database 132, where the instructions may include labeling of training examples. In some embodiments, panel training data may be updated iteratively using a feedback loop. As a non-limiting example, processor 108 may update panel training data iteratively through a feedback loop as a function of subject data 116, panel focuses 156a-n, ECG feature or feature vector, output of supplementary machine-learning module 144, language processing module, or any machine-learning models disclosed in this disclosure, or the like.

With continued reference to FIG. 1, in some embodiments, processor 108 may be configured to generate panel machine-learning models 152a-n of pre-operative panel machine-learning module 148. Processor 108 is configured to train each of plurality of panel machine-learning models 152a-n using each of plurality of sets of panel training data. In a non-limiting example, generating panel machine-learning models 152a-n may include training, retraining, or fine-tuning panel machine-learning models 152a-n using panel training data or updated panel training data. For example, and without limitation, processor 108 may train a first panel machine-learning model 152a using first set of panel training data and a second panel machine-learning model 152b using a second set of panel training data. Processor 108 is configured to generate a plurality of panel outputs 136a-n using each of plurality of panel machine-learning models 152a-n (i.e. trained or updated panel machine-learning models 152a-n).

With continued reference to FIG. 1, in a non-limiting example, if first panel machine-learning model 152a includes panel focus 156a of ejection fraction (EF), then panel output 136a of first panel machine-learning model 152a as a function subject data 116 and ECG data 120 may be an estimated ejection fraction characteristic. For the purposes of this disclosure, “estimated ejection fraction characteristic” is an estimated percentage of blood ejected from the left ventricle of the heart with each contraction. The EF characteristic may indicate, for example, an absolute estimate of the subject's EF (e.g. a particular value such as 50-percent, 45-percent, 40-percent, 35-percent, 30-percent, or another value) or a classification of the subject's estimated EF (e.g. normal EF greater than 50-percent, low EF between 35 and 50 percent, or very low EF below 35-percent).

With continued reference to FIG. 1, additionally, panel machine-learning model 152a-n that includes panel focus 156a-n related to pulmonary hypertension may be consistent with a model (e.g., diagnosis model or learning system) described in U.S. Non-provisional patent application Ser. No. 17/500,287, filed on Oct. 13, 2021, entitled “NONINVASIVE METHODS FOR DETECTION OF PULMONARY HYPERTENSION,” having an attorney docket number of 1518-029USU1 and U.S. Non-provisional patent application Ser. No. 18/771,678, filed on Jul. 12, 2024, entitled “APPARATUS AND METHOD FOR DETECTING HYPERTENSION ATTRIBUTES,” having an attorney docket number of 1518-127USU1, the entirety of each of which is incorporated herein as a reference. In a non-limiting example, algorithms may be developed using subject data 116; including ECG data 120, procedural measurements, physician notes, and subject demographics for the purpose of screening subjects for pulmonary hypertension. In order to distinguish between pulmonary hypertension and non-pulmonary hypertension patients for model training, each ECG may be paired with either a right heart catheterization (RHC) or an echocardiogram. Measurements derived from these procedures may be used to define cohorts. In a non-limiting example, subject data 116 including 65,994 unique patients (11,238 pulmonary hypertension and 54,756 non-pulmonary hypertension) of which 48% may be used in model training (e.g., panel machine-learning models 152a-n), 12% held-out for preliminary validation, and a final 40% held-out for testing. In some embodiments, panel machine-learning models 152a-n may use voltage-time information (e.g., ECG data 120) from 12-lead ECGs as inputs. Modeling techniques may include convolutional neural networks with differing structures such as using all 12 leads as a single input, groups of 3 leads as separate inputs, each lead converted to spectrogram, and combinations of these methods. Additionally, in some embodiments, two distinct preliminary models may be created, one in which the ECG was performed within a month of the patient's diagnosis (e.g., diagnostic model) and another in which the ECG was performed 6 to 18 months prior to the diagnosis date (e.g., pre-emptive model). In a non-limiting example, preliminary diagnostic model may include a convolutional neural network with residual connections incorporating the 12-lead single input. The updated diagnostic model may obtain an area under the curve (AUC) of 0.94 on the diagnostic validation and test sets, and may distinguish pulmonary hypertension 6 to 18 months prior to diagnosis with an AUC of 0.90 on the validation and test sets. In some aspects of the invention, disclosed herein may include methods including receiving voltage-time data (e.g., ECG data 120) of a subject, the voltage-time data including voltage data of a plurality of leads of an electrocardiogra generating a feature vector (e.g., ECG feature) from the voltage-time data, providing the feature vector to a pretrained learning system (e.g., panel machine-learning models 152a-n), and receiving from the pretrained learning system an indication of the presence or absence of pulmonary hypertension (e.g., panel output 136a-n) in the subject. Generating the feature vector may include generating a spectrogram based on the voltage data of the plurality of leads. In some embodiments, generating the feature vector may include grouping the voltage data of the plurality of leads into a plurality of subsets. In some embodiments, the voltage-time data of a subject may be received from an electrocardiogramarther embodiments, the voltage-time data of a subject may be received from an electronic medical record. In some embodiments, the method may further include providing an indication to an electronic health record system for storage in a health record associated with the subject. In some embodiments, the method may further include providing the indication to a computing node for display to a user.

With continued reference to FIG. 1, additionally, panel machine-learning model 152a-n that includes panel focus 156a-n related to coronary heart disease may be consistent with a model (e.g., learning system) described in U.S. Non-provisional patent application Ser. No. 18/642,012, filed on Apr. 22, 2024, entitled “SYSTEM AND A METHOD FOR ELECTROCARDIOGRAM OF COMPUTED TOMOGRAPHY-BASED HIGH CORONARY CALCIUM SCORE (CAC),” having an attorney docket number of 1518-006USU1, U.S. Non-provisional patent application Ser. No. 18/229,854, filed on Aug. 3, 2023, entitled “APPARATUS AND METHOD FOR DETERMINING A PATIENT SURVIVAL PROFILE USING ARTIFICIAL INTELLIGENCE-ENABLED ELECTROCARDIOGRAM (ECG),” having an attorney docket number of 1518-101USU1, and U.S. Non-provisional patent application Ser. No. 18/771,472, filed on Jul. 12, 2024, entitled “APPARATUS AND A METHOD FOR IDENTIFYING THE PROGRESSION OF CORONARY HEARTDISEASE” having an attorney docket number of 1518-128USU1, the entirety of which is incorporated herein as a reference. In some aspects of the invention, disclosed herein may include methods including receiving voltage-time data (e.g., ECG data 120) of a subject, the voltage-time data including voltage data of a plurality of leads of an electrocardiogram rating a feature vector (e.g., ECG feature) from the voltage-time data, wherein the feature vector may include a time-series of values indicating an amplitude of the voltage-time data for the plurality of leads, providing the feature vector to a pretrained learning system (e.g., panel machine-learning models 152a-n), and receiving from the pretrained learning system an indication of the level of CAC (e.g., panel outputs 136a-n) in the subject. Generating the feature vector may include generating a spectrogram based on the voltage data of the plurality of leads. In some embodiments, generating the feature vector may include grouping the voltage data of the plurality of leads into a plurality of subsets. In some embodiments, the pretrained learning system may be trained by receiving a training set of voltage-time data from a plurality of CAC patients.

With continued reference to FIG. 1, additionally, panel machine-learning model 152a-n that includes panel focus 156a-n related to atrial fibrillation may be consistent with a model for atrial fibrillation screening (e.g., atrial fibrillation detection neural network) described in U.S. Non-provisional patent application Ser. No. 17/275,276, filed on Mar. 11, 2021, entitled “NEURAL NETWORKS FOR ATRIAL FIBRILLATION SCREENING,” having an attorney docket number of 1518-002USU1, the entirety of which is incorporated herein as a reference. In some embodiments, processor 104 may be configured to record an ECG of a subject and to process the recording and, optionally, additional (auxiliary) data to generate an atrial fibrillation prediction (e.g., panel outputs 136a-n). The atrial fibrillation prediction can indicate a likelihood that the subject has experienced or is susceptible to developing atrial fibrillation. The prediction can be expressed as a probability or confidence score representing a probability or confidence that the subject has experienced or is susceptible to developing atrial fibrillation. In some implementations, the prediction may be expressed as a selection of a particular classification from multiple possible classifications that represents a most likely condition of the subject. For example, a binary classification can be made indicating whether there is at least a threshold probability or confidence level that the subject has experienced or is susceptible to developing atrial fibrillation. The atrial fibrillation prediction can identify this binary classification. As another example, the atrial fibrillation prediction can indicate a recommendation or selection of a monitoring or treatment option for the subject based on a likelihood of the subject having experienced or being susceptible to development of atrial fibrillation. For instance, the prediction can indicate a selection from the trinary of options to administer an anticoagulation medication to the patient, to not administer an anticoagulant but continue with periodic screenings, or to initiate continuous monitoring, e.g., using an implantable loop recorder ECG. Thresholds used for any decision boundaries can be extracted from retrospective analysis and can be presented with positive and negative predictive value (PPV and NPV).

With continued reference to FIG. 1, additionally, panel machine-learning model 152a-n that includes panel focus 156a-n related to ejection fraction may be consistent with a model (e.g., ejection-fraction prediction model) described in U.S. Non-provisional application Ser. No. 18/053,200, filed on Aug. 25, 2023, and entitled “ECG-BASED CARDIAC EJECTION-FRACTION SCREENING,” having an attorney docket number of 1518-001USC2, the entirety of which is incorporated herein as a reference. In some implementations, machine-learning models (e.g., panel machine-learning models 152a-n) such as neural networks are configured to process predictive inputs that characterize ECG data 120 and output an indication of the estimated ejection fraction of a subject (e.g., panel outputs 136a-n). The model may be trained to account for complex combinations of features that are not otherwise discernible to a human, but that have been determined (e.g., by an iterative training process) to correlate to particular ejection-fraction characteristics.

With continued reference to FIG. 1, additionally, panel machine-learning model 152a-n that includes panel focus 156a-n related to diastolic dysfunction may be consistent with a model or neural network described in U.S. Non-provisional patent application Ser. No. 18/592,680, filed on Mar. 1, 2024, entitled “APPARATUS AND METHOD FOR TRAINING AN ARTIFICIAL INTELLIGENCE-SUPPORTED DIAGNOSTIC ASSESSMENT TOOL,” having an attorney docket number of 1518-111USU1 and U.S. Non-provisional patent application Ser. No. 18/771,914, filed on Jul. 12, 2024, entitled “APPARATUS AND METHODS FOR IDENTIFYING ABNORMAL BIOMEDICAL FEATURES WITHIN IMAGES OF BIOMEDICAL DATA” having an attorney docket number of 1518-126USU1, the entirety of each of which is incorporated herein as a reference.

With continued reference to FIG. 1, additionally, panel machine-learning model 152a-n that includes panel focus 156a-n related to cardiac amyloidosis may be consistent with a model or learning system described in U.S. Non-provisional patent application Ser. No. 18/642,200, filed on Apr. 22, 2024, entitled “SYSTEM AND A METHOD FOR SCREENING FOR CARDIAC AMYLOIDOSIS BY ELECTROCARDIOGRAMY,” having an attorney docket number of 1518-004USU1, the entirety of which is incorporated herein as a reference.

With continued reference to FIG. 1, panel machine-learning models 152a-n may be consistent with various machine-learning models disclosed in U.S. Non-provisional patent application Ser. No. 18/750,336, filed on Jun. 21, 2024, entitled “SYSTEMS AND METHODS FOR TRACKING CARDIAC VALUES” having an attorney docket number of 1518-125USU1, the entirety of each of which are incorporated herein as references.

With continued reference to FIG. 1, in some embodiments, panel machine-learning models 152a-n may include a generative artificial intelligence. In some embodiments, processor 104 may implement one or more aspects of “generative artificial intelligence (AI),” a type of AI that uses machine learning algorithms to create, establish, or otherwise generate data such as, without limitation, panel outputs 136a-n, and/or the like in any data structure as described herein (e.g., text, image, video, audio, among others) that is similar to one or more provided training examples. In an embodiment, machine learning module described herein may generate one or more generative machine learning models that are trained on one or more set of panel training data. One or more generative machine learning models may be configured to generate new examples that are similar to the training data of the one or more generative machine learning models but are not exact replicas; for instance, and without limitation, data quality or attributes of the generated examples may bear a resemblance to the training data provided to one or more generative machine learning models, wherein the resemblance may pertain to underlying patterns, features, or structures found within the provided training data.

With continued reference to FIG. 1, in some cases, generative machine learning models may include one or more generative models. As described herein, “generative models” refers to statistical models of the joint probability distribution P(X,Y) on a given observable variable x, representing features or data that can be directly measured or observed (e.g., subject data 116, ECG data 120, ECG feature, or the like) and target variable y, representing the outcomes or labels that one or more generative models aims to predict or generate (e.g., panel outputs 136a-n). In some cases, generative models may rely on Bayes theorem to find joint probability; for instance, and without limitation, Naïve Bayes classifiers may be employed by computing device to categorize input data such as, without limitation, subject data 116 into different subject cohorts such as, without limitation, age, gender, or the like of subject cohorts.

In a non-limiting example, and with continued reference to FIG. 1, one or more generative machine learning models may include one or more Naïve Bayes classifiers generated, by computing device using a Naïve bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing Device may then calculate a likelihood table by calculating probabilities of different data entries and classification labels. Computing device may utilize a naïve Bayes equation to calculate a posterior probability for each class. A class containing the highest posterior probability is the outcome of prediction.

With continued reference to FIG. 1, although Naïve Bayes classifier may be primarily known as a probabilistic classification algorithm; however, it may also be considered a generative model described herein due to its capability of modeling the joint probability distribution P(X,Y) over observable variables X and target variable Y. In an embodiment, Naïve Bayes classifier may be configured to make an assumption that the features X are conditionally independent given class label Y, allowing generative model to estimate the joint distribution as P(X,Y)=P(Y)ΠiP(Xi|Y), wherein P(Y) may be the prior probability of the class, and P(Xᵢ|Y) is the conditional probability of each feature given the class. One or more generative machine learning models containing Naïve Bayes classifiers may be trained on labeled training data, estimating conditional probabilities P(Xᵢ|Y) and prior probabilities P(Y) for each class; for instance, and without limitation, using techniques such as Maximum Likelihood Estimation (MLE). One or more generative machine learning models containing Naïve Bayes classifiers may select a class label y according to prior distribution P(Y), and for each feature Xᵢ, sample at least a value according to conditional distribution P(Xᵢ|y). Sampled feature values may then be combined to form one or more new data instance with selected class label y. In a non-limiting example, one or more generative machine learning models may include one or more Naïve Bayes classifiers to generate new examples of panel outputs 136a-n based on subject cohorts, wherein the models may be trained using training data containing a plurality of features e.g., data features if any or simply “features of subject data 116 or ECG data 120”, and/or the like as input correlated to a plurality of labeled classes e.g., subject cohorts as output.

With continued reference to FIG. 1, in some cases, one or more generative machine learning models may include generative adversarial network (GAN). As used in this disclosure, a “generative adversarial network” is a type of artificial neural network with at least two sub models (e.g., neural networks), a generator, and a discriminator, that compete against each other in a process that ultimately results in the generator learning to generate new data samples, wherein the “generator” is a component of the GAN that learns to create hypothetical data by incorporating feedbacks from the “discriminator” configured to distinguish real data from the hypothetical data. In some cases, generator may learn to make discriminator classify its output as real. In an embodiment, discriminator may include a supervised machine learning model while generator may include an unsupervised machine learning model as described in further detail with reference to FIG. 5.

With continued reference to FIG. 1, in an embodiment, discriminator may include one or more discriminative models, i.e., models of conditional probability P(Y|X=x) of target variable Y, given observed variable X. In an embodiment, discriminative models may learn boundaries between classes or labels in given training data. In a non-limiting example, discriminator may include one or more classifiers as described in further detail below with reference to FIG. 5 to distinguish between different categories e.g., correct vs. incorrect, or states e.g., TRUE vs. FALSE within the context of generated data such as, without limitations, panel outputs 136a-n, and/or the like. In some cases, computing device may implement one or more classification algorithms such as, without limitation, Support Vector Machines (SVM), Logistic Regression, Decision Trees, and/or the like to define decision boundaries.

In a non-limiting example, and with continued reference to FIG. 1, generator of GAN may be responsible for creating synthetic data that resembles real panel outputs 136a-n. In some cases, GAN may be configured to receive subject data 116 such as, without limitation, ECG data 120, as input and generates corresponding panel outputs 136a-n containing information describing or evaluating the performance of one or more ECG signals. On the other hand, discriminator of GAN may evaluate the authenticity of the generated content by comparing it to real panel outputs 136a-n, for example, discriminator may distinguish between genuine and generated content and providing feedback to generator to improve the model performance.

With continued reference to FIG. 1, in other embodiments, one or more generative models may also include a variational autoencoder (VAE). As used in this disclosure, a “variational autoencoder” is an autoencoder (i.e., an artificial neural network architecture) whose encoding distribution is regularized during the model training process in order to ensure that its latent space includes desired properties allowing new data sample generation. In an embodiment, VAE may include a prior and noise distribution respectively, trained using expectation-maximization meta-algorithms such as, without limitation, probabilistic PCA, sparse coding, among others. In a non-limiting example, VEA may use a neural network as an amortized approach to jointly optimize across input data and output a plurality of parameters for corresponding variational distribution as it maps from a known input space to a low-dimensional latent space. Additionally, or alternatively, VAE may include a second neural network, for example, and without limitation, a decoder, wherein the “decoder” is configured to map from the latent space to the input space.

In a non-limiting example, and with continued reference to FIG. 1, VAE may be used by computing device to model complex relationships between subject data 116 e.g., ECG data 120. In some cases, VAE may encode input data into a latent space, capturing panel outputs 136a-n. Such encoding process may include learning one or more probabilistic mappings from observed subject data 116 and/or ECG data 120 to a lower-dimensional latent representation. Latent representation may then be decoded back into the original data space, therefore reconstructing the subject data 116 and/or ECG data 120. In some cases, such decoding process may allow VAE to generate new examples or variations that are consistent with the learned distributions.

With continued reference to FIG. 1, processor 108 may configure generative machine learning models to analyze input data such as, without limitation, ECG data 120 to one or more predefined templates representing correct panel outputs 136a-n, thereby allowing processor 108 to identify discrepancies or deviations from panel outputs 136a-n. In some cases, computing device may be configured to pinpoint specific errors in ECG data 120 or any other aspects of the subject data 116. In a non-limiting example, processor 108 may be configured to implement generative machine learning models to incorporate additional models to detect any data. In some cases, errors may be classified into different categories or severity levels. In a non-limiting example, some errors may be considered minor, and generative machine learning model such as, without limitation, GAN may be configured to generate panel outputs 136a-n contain only slight adjustments while others may be more significant and demand more substantial corrections. In some embodiments, computing device may be configured to flag or highlight ECG features, which affects a subject's health condition significantly using one or more generative machine learning models described herein. In some cases, one or more generative machine learning models may be configured to generate and output indicators such as, without limitation, visual indicator, audio indicator, and/or any other indicators as described above. Such indicators may be used to signal the detected error described herein.

With continued reference to FIG. 1, other exemplary embodiments of generative machine learning models may include, without limitation, long short-term memory networks (LSTMs), (generative pre-trained) transformer (GPT) models, mixture density networks (MDN), and/or the like. As an ordinary person skilled in the art, upon reviewing the entirety of this disclosure, will be aware of various generative machine learning models may be used to generate panel outputs 136a-n.

With continued reference to FIG. 1, in a further non-limiting embodiment, machine learning module may be further configured to generate a multi-model neural network that combines various neural network architectures described herein. In a non-limiting example, multi-model neural network may combine LSTM for time-series analysis with GPT models for natural language processing. Such fusion may be applied by computing device to generate panel outputs 136a-n. In some cases, multi-model neural network may also include a hierarchical multi-model neural network, wherein the hierarchical multi-model neural network may involve a plurality of layers of integration; for instance, and without limitation, different models may be combined at various stages of the network. Convolutional neural network (CNN) may be used for image feature extraction, followed by LSTMs for sequential pattern recognition, and a MDN at the end for probabilistic modeling. Other exemplary embodiments of multi-model neural network may include, without limitation, ensemble-based multi-model neural network, cross-modal fusion, adaptive multi-model network, among others. As an ordinary person skilled in the art, upon reviewing the entirety of this disclosure, will be aware of various generative machine learning models may be used to [limitation/processing step] described herein. As an ordinary person skilled in the art, upon reviewing the entirety of this disclosure, will be aware of various multi-model neural network and combination thereof that may be implemented by apparatus 100 in consistent with this disclosure.

With continued reference to FIG. 1, panel machine-learning models 152a-n may include a large language model (LLM) 160. A “large language model,” as used herein, is a deep learning data structure that can recognize, summarize, translate, predict and/or generate text and other content based on knowledge gained from massive datasets. In a non-limiting example, panel machine-learning models 152a-n may generate panel outputs 136a-n that is in natural human language. Large language models 160 may be trained on large sets of data. Training sets may be drawn from diverse sets of data such as, as non-limiting examples, articles, research papers, unstructured data, electronic records, and the like. In some embodiments, training sets may include a variety of subject matters, such as, as nonlimiting examples, medical report documents, electronic health records, entity documents, emails, user communications, research articles, and the like. In some embodiments, training sets of LLM 160 may include information from one or more public or private databases. As a non-limiting example, training sets may include databases associated with an entity. In some embodiments, training sets may include portions of documents associated with the electronic records correlated to examples of outputs (e.g., panel outputs 136a-n). In an embodiment, LLM 160 may include one or more architectures based on capability requirements of LLM 160. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 1, in some embodiments, LLM 160 may be generally trained. As used in this disclosure, a “generally trained” LLM is LLM that is trained on a general training set including a variety of subject matters, data sets, and fields. In some embodiments, LLM 160 may be initially generally trained. Additionally, or alternatively, LLM 160 may be specifically trained. As used in this disclosure, a “specifically trained” LLM is LLM that is trained on a specific training set, wherein the specific training set includes data including specific correlations for the LLM 160 to learn. As a non-limiting example, LLM 160 may be generally trained on a general training set, then specifically trained on a specific training set. In an embodiment, specific training of LLM 160 may be performed using a supervised machine learning process. In some embodiments, generally training LLM 160 may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as LLM 160 may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as LLM 160 may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 1, in some embodiments, LLM 160 may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. LLM 160 may include a text prediction based algorithm configured to receive an article and apply a probability distribution to the words already typed in a sentence to work out the most likely word to come next in augmented articles. For example, if some words that have already been typed are “First recommended”, then it may be highly likely that the word “treatment” will come next. LLM 160 may output such predictions by ranking words by likelihood or a prompt parameter. For the example given above, LLM 160 may score “treatment” as the most likely, “medication” as the next most likely, “alternative” or “drug” next, and the like. LLM 160 may include an encoder component and a decoder component.

With continued reference to FIG. 1, LLM 160 may include a transformer architecture. In some embodiments, encoder component of LLM 160 may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, LLM 160 and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, LLM 160 may predict the next word by searching for a set of positions in a source sentence where the most relevant information is concentrated. LLM 160 may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

With continued reference to FIG. 1, attention mechanism may include, without limitation, generalized attention self-attention, multi-head attention, additive attention, global attention, and the like. In generalized attention, when a sequence of words or an image is fed to LLM 160, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, LLM 160 may pick up particular parts at different positions in the input sequence and over time compute an initial composition of the output sequence. In multi-head attention, LLM 160 may include a transformer model of an attention mechanism. Attention mechanisms, as described above, may provide context for any position in the input sequence. For example, if the input data is a natural language sentence, the transformer does not have to process one word at a time. In multi-head attention, computations by LLM 160 may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), LLM 160 may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, LLM 160 may either attend to all source words or predict the target sentence, thereby attending to a smaller subset of words.

With continued reference to FIG. 1, multi-headed attention in encoder may apply a specific attention mechanism called self-attention. Self-attention allows models such as LLM 160 or components thereof to associate each word in the input, to other words. As a non-limiting example, LLM 160 may learn to associate the word “you”, with “how” and “are”. It's also possible that LLM 160 learns that words structured in this pattern are typically a question and to respond appropriately. In some embodiments, to achieve self-attention, input may be fed into three distinct fully connected neural network layers to create query, key, and value vectors. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens”.

With continued reference to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

With continued reference to FIG. 1, decoder may take this output and add it to the decoder inputs. Decoder may continue decoding until a token is predicted. Decoder may stop decoding once it predicts an end token.

Continuing to refer to FIG. 1, in some embodiment, decoder may be stacked N layers high, with each layer taking in inputs from the encoder and layers before it. Stacking layers may allow LLM 160 to learn to extract and focus on different combinations of attention from its attention heads.

With continued reference to FIG. 1, LLM 160 may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device 128. User device 128 may be any computing device that is used by a user. As non-limiting examples, user device 128 may include desktops, laptops, smartphones, tablets, and the like. In some embodiments, input may include any set of data associated with subject data 116, ECG data 120, panel focus 156a-n, panel outputs 136a-n, or the like.

With continued reference to FIG. 1, LLM 160 may generate at least one annotation as an output. At least one annotation may be any annotation as described herein. In some embodiments, LLM 160 may include multiple sets of transformer architecture as described above. Output may include a textual output. A “textual output,” for the purposes of this disclosure is an output including a string of one or more characters. Textual output may include, for example, a plurality of annotations for unstructured data. In some embodiments, textual output may include a phrase or sentence answering to a query from a user. In some embodiments, textual output may include a sentence or plurality of sentences of panel outputs 136a-n.

With continued reference to FIG. 1, in some embodiments, panel outputs 136a-n may further include a pre-operative optimization output. For the purposes of this disclosure, a “pre-operative optimization output” is a panel output that is related to the process of improving a subject's health status before undergoing surgery to enhance their outcomes and reduce the risk of complications or as an alternative to the surgery. As a non-limiting example, pre-operative optimization output may include suggestion for nutritional optimization, physical therapy, medications, non-surgical surgery, or the like. In some embodiments, pre-operative optimization output may be stored in subject database 132. In some embodiments, pre-operative optimization output may be retrieved from subject database 132. In some embodiments, user may manually input pre-operative optimization output.

With continued reference to FIG. 1, in some embodiments, panel machine-learning models 152a-n may include an optimization output machine-learning model 164. In some embodiments, processor 108 may generate pre-operative optimization output of panel outputs 136a-n using an optimization output machine-learning model 164. In some embodiments, processor 108 may be configured to generate optimization output training data. In a non-limiting example, optimization output training data may include correlations between exemplary subject data, exemplary panel focuses and exemplary pre-operative optimization outputs. In some embodiments, optimization output training data may be stored in subject database 132. In some embodiments, optimization output training data may be received from one or more users, subject database 132, external computing devices, and/or previous iterations of processing. As a non-limiting example, optimization output training data may include instructions from a user, who may be an expert user, a past user in embodiments disclosed herein, or the like, which may be stored in memory and/or stored in subject database 132, where the instructions may include labeling of training examples. In some embodiments, optimization output training data may be updated iteratively using a feedback loop. As a non-limiting example, processor 108 may update optimization output training data iteratively through a feedback loop as a function of subject data 116, ECG data 120, output of any machine-learning models described in this disclosure, or the like. In some embodiments, processor 108 may be configured to generate optimization output machine-learning model 164. In a non-limiting example, generating optimization output machine-learning model 164 may include training, retraining, or fine-tuning optimization output machine-learning model 164 using optimization output training data or updated optimization output training data. In some embodiments, processor 108 may be configured to generate pre-operative optimization output of panel outputs 136a-n using optimization output machine-learning model 164 (i.e. trained or updated optimization output machine-learning model 164). In some embodiments, processor 108 may generate pre-operative optimization output using the combination of optimization output machine-learning model 164 and large language model 160.

With continued reference to FIG. 1, in some embodiments, processor 108 may be configured to generate panel outputs 136a-n using a panel lookup table. For the purposes of this disclosure, a “panel lookup table” is a lookup table that generates panel outputs. A “lookup table,” for the purposes of this disclosure, is an array of data that maps input values to output values. The lookup table may be used to replace a runtime computation with an array indexing operation. In an embodiment, the lookup table may include interpolation. For the purposes of this disclosure, an “interpolation” refers to a process for estimating values that lie between known data. As a non-limiting example, the lookup table may include an output value for each of input values. When the lookup table does not define the input values, then the lookup table may estimate the output values based on the nearby table values. In another embodiment, the lookup table may include an extrapolation. For the purposes of this disclosure, an “extrapolation” refers to a process for estimating values that lie beyond the range of known data. As a non-limiting example, the lookup table may linearly extrapolate the nearest data to estimate an output value for an input beyond the data. As a non-limiting example, processor 108 may be configured to “lookup” given subject data 116 and/or ECG data 120 to find a corresponding panel output 136a-n.

With continued reference to FIG. 1, in some embodiments, apparatus 100 may further include a supplementary machine-learning module 144. For the purposes of this disclosure, a “supplementary machine-learning module” is a component or package that provides functionalities for building, training, and deploying machine-learning models for analyzing subject data. In some embodiments, outputs of any machine-learning models in supplementary machine-learning module 144 may be fed into pre-operative panel machine-learning module 148, updating plurality of sets of panel training data of plurality of panel machine-learning models 152a-n.

With continued reference to FIG. 1, in some embodiments, supplementary machine-learning module 144 may include a cohort classifier 168. Cohort classifier 168 may be consistent with any classifier discussed in this disclosure. For the purposes of this disclosure, a “cohort classifier” is a data structure representing and/or using a mathematical model, neural net, or program generated by a machine learning algorithm known as a “classification algorithm,” as described in further detail below, that sorts inputs into one or more cohorts, outputting the cohorts associated therewith. In some embodiments, subject or subject data 116 may be classified to a subject cohort using a cohort classifier 168 of a supplementary machine-learning module 144. For the purposes of this disclosure, a “subject cohort” is a set of associative characteristic of a subject. As a non-limiting example, processor 108 may classify subject data 116 into subject cohort as a function of a subject's age, gender, medical history, comorbid conditions, lifestyle factors, smoking, alcohol consumption, obesity, nutritional status, genetic factor, or the like. In some embodiments, processor 108 may generate cohort training data. In some embodiments, cohort classifier 168 may be trained on cohort training data, wherein the cohort training data may include correlations between exemplary subject data and exemplary subject cohorts. In some embodiments, a subject may be classified to a subject cohort and processor 108 may generate panel output 136a-n based on the subject cohort and the resulting output may be used to update panel training data. In some embodiments, generating training data and training machine-learning models may be simultaneous.

With continued reference to FIG. 1, processor 108 may use a machine learning module, such as pre-operative panel machine-learning module 148, supplementary machine-learning module 144, or the like, to implement one or more algorithms or generate one or more machine-learning models, such as panel machine-learning models 152a-n, cohort classifier 168, or the like and calculate data as described herein. However, the machine learning module is exemplary and may not be necessary to generate one or more machine learning models and perform any machine learning described herein. In one or more embodiments, one or more machine-learning models may be generated using training data. Training data may include inputs and corresponding predetermined outputs so that a machine-learning model may use correlations between the provided exemplary inputs and outputs to develop an algorithm and/or relationship that then allows machine-learning model to determine its own outputs for inputs. Training data may contain correlations that a machine-learning process may use to model relationships between two or more categories of data elements. Exemplary inputs and outputs may come from database, such as subject database 132 described in this disclosure, or be provided by a user. In other embodiments, a machine-learning module may obtain a training set by querying a communicatively connected database that includes past inputs and outputs. Training data may include inputs from various types of databases, resources, and/or user inputs and outputs correlated to each of those inputs so that a machine-learning model may determine an output. Correlations may indicate causative and/or predictive links between data, such as subject data 116 and panel outputs 136a-n, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements. Machine learning module may be used to generate a machine learning model and/or any other machine learning model using training data. Machine learning model may be trained by correlated inputs and outputs of training data. Training data may be data sets that have already been converted from raw data whether manually, by machine, or any other method. Training data may be stored in database. Training data may also be retrieved from database.

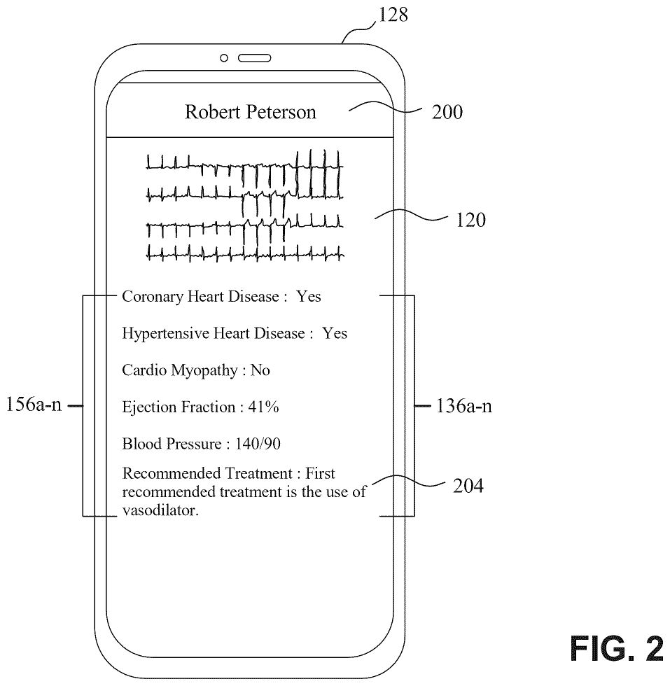

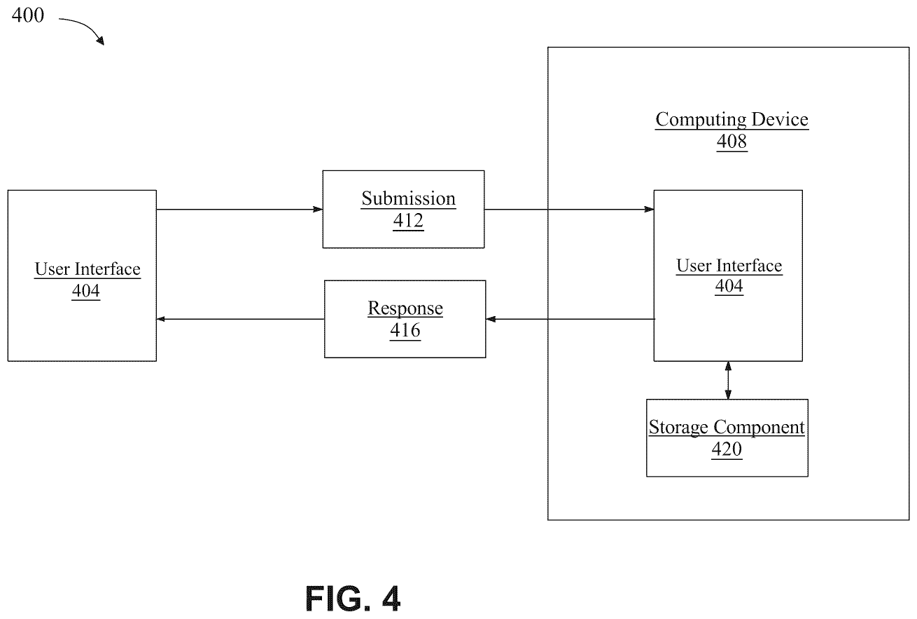

With continued reference to FIG. 1, memory 112 contains instructions configuring processor 108 to generate a pre-operative data structure 104 as a function of plurality of panel outputs 136a-n. For the purposes of this disclosure, a “pre-operative data structure” is a structured organization of data related to a plurality of panel outputs related to pre-operative determinations. In some embodiments, pre-operative data structure 104 may include a form of text, graph, trend line, chart, audio, animation, image, video, and the like. In some embodiments, pre-operative data structure 104 may include various format. As a non-limiting example, pre-operative data structure 104 may include PDF, DOC, XLS, HTML, PNC, JPEG, BMP, TIFF, MP4, or the like. As another non-limiting example, pre-operative data structure 104 may include a form of a chatbot. For the purposes of this disclosure, “chatbot” is an artificial intelligence (AI) program designed to simulate human conversation or interaction through text, voice-based or image-based communication. As a non-limiting example, user may input panel focus 156a-n into chatbot and panel output 136a-n may be outputted according to panel focus 156a-n. Chatbot disclosed herein is further described with respect to FIG. 4. In some embodiments, pre-operative data structure 104 may provide a summarization, representation, or otherwise abstraction of panel outputs 136a-n. In a non-limiting example, pre-operative data structure 104 may include a comprehensive report on panel outputs 136a-n. In another non-limiting example, pre-operative data structure 104 may include a list of panel outputs 136a-n with panel focuses 156a-n. For example, and without limitation, pre-operative data structure 104 may include a first panel focus 156a following with a first panel output 136a, a second panel focus 156b following with a second panel output 136b and n number of panel focus 156n following with n number of panel output 136n. Exemplary pre-operative data structure 104 is illustrated in FIG. 2. In another non-limiting example, pre-operative data structure 104 may include panel outputs 136a-n and associated subject data 116 or ECG data 120, or the like. In some embodiments, pre-operative data structure 104 may be stored in pre-operative data structure 104. In some embodiments, pre-operative data structure 104 may be retrieved from pre-operative data structure 104. In some embodiments, user may manually input pre-operative data structure 104. In some embodiments, pre-operative data structure 104 may be retrieved from a cloud storage.

With continued reference to FIG. 1, in some embodiments, generating pre-operative data structure 104 may include retrieving a data structure template. For the purposes of this disclosure, a “data structure template” is a pre-designed structure or framework of a pre-operative data structure. In a non-limiting example, data structure template may include every panel focuses 156a-n that can be asked to a subject as a pre-operative panel. In some embodiments, data structure template may be consistent with pre-operative data structure 104 except template form fields of data structure template are not filled which is described further in detail below. In some embodiments, data structure template may be stored in subject database 132. In some embodiments, data structure template may be retrieved from subject database 132. In some embodiments, user may manually input data structure template.

With continued reference to FIG. 1, in some embodiments, generating pre-operative data structure 104 may include identifying a template form field of data structure template. For the purposes of this disclosure, a “template form field” is a specific area within a data structure template, which is designated for an input of specific types of information. As a non-limiting example, template form field may include text fields, checkboxes, and the like. In an embodiment, template form field may include personal data form field, wherein personal information associated with a subject from subject data 116 can be put in the personal data form field. As a non-limiting example, personal data form field may include form field for subject's name, date of birth, age, weight, or the like. In another embodiment, template form field may include ECG data form field, where ECG data 120 can be put in the ECG data form field. In another embodiment, template form field may include panel output form field, wherein panel outputs 136a-n can be put in the panel output form field. In a non-limiting example, panel output form field may be adjacent to panel focuses 156a-n in data structure template so that each panel outputs 136a-n can be shown as an answer to its related panel focuses 156a-n.

With continued reference to FIG. 1, in some embodiments, processor 108 may automatically fill template form field of data structure template using a template form field machine learning model 172. In some embodiments, supplementary machine-learning module 144 may include template form field machine learning model 172. For the purposes of this disclosure, a “template form field machine learning model” is a machine learning model that fills at least a template form field of a data structure template. Template form field machine learning model 172 disclosed herein may be consistent with a machine learning model disclosed with respect to FIG. 5 Template form field machine learning model 172 may be trained with template form field training data. For the purposes of this disclosure, “template form field training data” is training data that is used to train template form field machine learning model. The training data disclosed herein is further disclosed with respect to FIG. 5. In some embodiments, template form field machine learning model 172 may be trained with template form field training data that correlates panel outputs 136a-n to template form field of data structure template. In some embodiments, template form field training data may be received from a user, subject database 132, external computing devices, and/or previous iterations of processing. As a non-limiting example, template form field training data may include instructions from a user, including an expert user or past user, stored in subject database 132, where the instructions may include labeling examples. In some embodiments, template form field machine learning model 172 may be implemented with data mapping process. For the purposes of this disclosure, “data mapping” is the process of defining the relationships between data elements in different entities. In some embodiments, data mapping may include mapping fields, defining the data types and formats to be used, and identifying any transformations or conversions that are necessary.

With continued reference to FIG. 1, in some embodiments, processor 108 may be configured to transmit pre-operative data structure 104 to a user device 128. In some embodiments, processor 108 may be configured to generate a user interface displaying pre-operative data structure 104, subject data 116, ECG data 120, panel outputs 136a-n, and the like. For the purposes of this disclosure, a “user interface” is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof and the like. In some embodiments, user interface may operate on and/or be communicatively connected to a decentralized platform, metaverse, and/or a decentralized exchange platform associated with the user. In some embodiments, a user may interact with the use interface using a computing device distinct from and communicatively connected to at least a processor 108. For example, a smart phone, smart, tablet, or laptop operated by a user. In an embodiment, user interface may include a graphical user interface. A “graphical user interface,” as used herein, is a graphical form of user interface that allows users to interact with electronic devices. In some embodiments, GUI may include icons, menus, other visual indicators or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access.

With continued reference to FIG. 1, in some embodiments, processor 108 may be configured to receive a user prompt. For the purposes of this disclosure, a “user prompt” is a data input or command provided by a user to a computer system, software application, or interface. As a non-limiting example, user may input user prompt using chatbot, user interface of user device 128, or the like. In some embodiments, user prompt may include a query expressed in natural language, code, or any other format. As a non-limiting example, user prompt may include query for a pre-operative data structure 104 and processor 108 may generate pre-operative data structure 104 as a reception of a query. As another non-limiting example, user prompt may include query for a specific panel output 136a-n and processor 108 may generate panel output 136a-n, then display to user device 128 as pre-operative data structure 104, as a reception of query. In some embodiments, user prompt may include panel focus 156a-n. As a non-limiting example, user prompt may include question related to a specific cardiac disease and processor 108 may analyze user prompt and determine the specific cardiac disease as panel focus 156a-n. In some embodiments, user prompt may include text, audio, gestures, button clicks, image, video, or the like. In some embodiments, user prompt may include feedback related to pre-operative data structure 104. In some embodiments, processor 108 may store user prompt in subject database 132. In some embodiments, processor 108 may retrieve user prompt from subject database 132.

With continued reference to FIG. 1, in some embodiments, processor 108 may use a language processing module to find at least a keyword. In some embodiments, processor 108 may use at least a keyword found in user prompt as panel focus 156a-n. The language processing module may be configured to extract, from user prompt, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

With continued reference to FIG. 1, language processing module may operate to produce a language processing model. Language processing model may include a program automatically generated by processor 108 and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

With continued reference to FIG. 1, language processing module may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs, as used herein, are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

With continued reference to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

With continued reference to FIG. 1, language processing module may use a corpus of documents to generate associations between language elements in a language processing module may then use such associations to analyze words extracted from one or more documents and determine that the one or more documents indicate significance of a category. In an embodiment, language module and/or processor 108 may perform this analysis using a selected set of significant documents, such as documents identified by one or more experts as representing good information; experts may identify or enter such documents via graphical user interface or may communicate identities of significant documents according to any other suitable method of electronic communication, or by providing such identity to other persons who may enter such identifications into processor 108. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, processor 108 may automatically obtain the document using such an identifier, for instance by submitting a request to a database or compendium of documents such as JSTOR as provided by Ithaka Harbors, Inc. of New York.

With continued reference to FIG. 1, in some embodiments, the use of machine-learning modules (e.g., pre-operative panel machine-learning module 148, supplementary machine-learning module 144, language processing module, or the like) may improve identifying hidden patterns, correlations, or anomalies that may be difficult for humans or existing systems to detect; for instance, determining panel outputs 136a-n. This may enhance the accuracy and efficiency of decision-making processes. In another non-limiting example, pre-operative panel machine-learning module 148, supplementary machine-learning module 144, language processing module, or the like may enable the automation of tasks that would otherwise require significant manual effort or expertise; for instance, determining ECG feature, determining panel outputs 136a-n, generating pre-operative data structure 104, or the like. By leveraging pre-operative panel machine-learning module 148, supplementary machine-learning module 144, language processing module, or the like, apparatus 100 may automatically process, analyze, and interpret large volumes of data, reducing the time and resources required for manual analysis and improving the overall efficiency of the technical process; for instance, analyzing subject data 116, ECG data 120, user prompt, or the like. In another non-limiting example, the use of pre-operative panel machine-learning module 148, supplementary machine-learning module 144, language processing module, or the like may enable analyzing data and making decisions in real-time or near real-time, allowing processor 108 to respond quickly to changing conditions or dynamic environments; for instance, analyzing ECG data 120 and generating panel outputs 136a-n. In another non-limiting example, pre-operative panel machine-learning module 148, supplementary machine-learning module 144, language processing module, or the like may learn from individual user preferences, behaviors, or feedback to personalize and customize the technical process; for instance, using user prompt or generating panel outputs 136a-n using subject cohorts. For example, and without limitation, pre-operative panel machine-learning module 148, supplementary machine-learning module 144, language processing module, or the like can analyze subject data 116, ECG data 120, and the like to provide tailored recommendations, optimize settings, or adapt the process to individual needs; for instance, generating panel outputs 136a-n as a reception of user prompt, classifying subject data to subject cohorts and generating panel outputs 136a-n using subject cohorts. This may enhance user experience and satisfaction. In another non-limiting example, pre-operative panel machine-learning module 148, supplementary machine-learning module 144, language processing module, or the like may learn from historical data and generate predictive models that forecast future outcomes or trends predict events, identify potential failures or risks, optimize resource allocation, anticipate user behavior or determine optimal solutions. This proactive approach may enable better planning, resource management, and decision-making. These may be consistent with any machine learning model described in this disclosure.

Referring now to FIG. 2, an exemplary pre-operative data structure 104 displayed on a user device 128 is illustrated. As a non-limiting example, user device 128 may include a laptop, desktop, tablet, mobile phone, smart phone, smart watch, kiosk, smart headset, or things of the like. In some embodiments, processor 108 may generate user interface to display pre-operative data structure 104. As a non-limiting example, pre-operative data structure 104 may include ECG data 120 as shown in FIG. 2. As another non-limiting example, pre-operative data structure 104 may include subject data 116; for instance, name 200 of a subject is illustrated in FIG. 2. As another non-limiting example, pre-operative data structure 104 may include panel focuses 156a-n as illustrated in FIG. 2. As another non-limiting example, pre-operative data structure 104 may include panel outputs 136a-n associated to panel focuses 156a-n as illustrated in FIG. 2. As another non-limiting example, pre-operative data structure 104 may include pre-operative optimization output 204 as illustrated in FIG. 2.

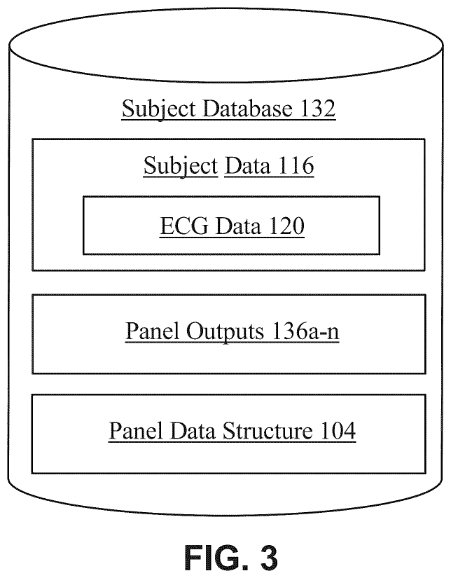

Referring now to FIG. 3, a block diagram of an exemplary subject database 132 is illustrated. In some embodiments, subject database 132 may store information related to subject data 116. As a non-limiting example, subject database 132 may store ECG data, ECG feature, ECG feature training data, input and output of ECG feature machine-learning model 140, or the like. In some embodiments, subject database 132 may store information related to panel outputs 136a-n. As a non-limiting example, subject database 132 may store information panel focuses 156a-n, panel training data, input and output of panel machine-learning models 152a-n, pre-operative optimization output, optimization output training data, input and output of optimization output machine-learning model 164, subject cohort, cohort training data, input and output of cohort classifier 168, image training data, input and output of image classifier, input and output of large language model 160, language training data, or the like. In some embodiments, subject database 132 may store information related to pre-operative data structure 104. As a non-limiting example, subject database 132 may store template form field training data, input and output of template form field machine-learning model 172, data structure template, or the like. In some embodiments, subject database 132 may store information related to user prompt. As a non-limiting example, subject database 132 may store at least a keyword from user prompt, input and output of language processing module, or the like.

Referring to FIG. 4, a chatbot system 400 is schematically illustrated. According to some embodiments, a user interface 404 may be communicative with a computing device 408 that is configured to operate a chatbot. In some cases, user interface 404 may be local to computing device 408. Alternatively or additionally, in some cases, user interface 404 may remote to computing device 408 and communicative with the computing device 408, by way of one or more networks, such as without limitation the internet. Alternatively or additionally, user interface 404 may communicate with user device 408 using telephonic devices and networks, such as without limitation fax machines, short message service (SMS), or multimedia message service (MMS). Commonly, user interface 404 communicates with computing device 408 using text-based communication, for example without limitation using a character encoding protocol, such as American Standard for Information Interchange (ASCII). Typically, a user interface 404 conversationally interfaces a chatbot, by way of at least a submission 412, from the user interface 408 to the chatbot, and a response 416, from the chatbot to the user interface 404. As a non-limiting example, response 416 may include panel outputs 136a-n. In many cases, one or both of submission 412 and response 416 are text-based communication. Alternatively or additionally, in some cases, one or both of submission 412 and response 416 are audio-based communication.

Continuing in reference to FIG. 4, a submission 412 once received by computing device 408 operating a chatbot, may be processed by a processor. In some embodiments, processor processes a submission 412 using one or more of keyword recognition, pattern matching, and natural language processing. In some embodiments, processor employs real-time learning with evolutionary algorithms. In some cases, processor may retrieve a pre-prepared response from at least a storage component 420, based upon submission 412. Alternatively or additionally, in some embodiments, processor communicates a response 416 without first receiving a submission 412, thereby initiating conversation. In some cases, processor communicates an inquiry to user interface 404; and the processor is configured to process an answer to the inquiry in a following submission 412 from the user interface 404. In some cases, an answer to an inquiry present within a submission 412 from a user device 404 may be used by computing device 408 as an input to another function.

With continued reference to FIG. 4, a chatbot may be configured to provide a user with a plurality of options as an input into the chatbot. Chatbot entries may include multiple choice, short answer response, true or false responses, and the like. A user may decide on what type of chatbot entries are appropriate. In some embodiments, the chatbot may be configured to allow the user to input a freeform response into the chatbot. The chatbot may then use a decision tree, data base, or other data structure to respond to the users entry into the chatbot as a function of a chatbot input. As used in the current disclosure, “chatbot input” is any response that a user inputs into a chatbot as a response to a prompt or question. As a non-limiting example, chatbot input may include a user prompt as described with respect to FIG. 1.

With continuing reference to FIG. 4, computing device 408 may be configured to the respond to a chatbot input using a decision tree. A “decision tree,” as used in this disclosure, is a data structure that represents and combines one or more determinations or other computations based on and/or concerning data provided thereto, as well as earlier such determinations or calculations, as nodes of a tree data structure where inputs of some nodes are connected to outputs of others. Decision tree may have at least a root node, or node that receives data input to the decision tree, corresponding to at least a candidate input into a chatbot. Decision tree has at least a terminal node, which may alternatively or additionally be referred to herein as a “leaf node,” corresponding to at least an exit indication; in other words, decision and/or determinations produced by decision tree may be output at the at least a terminal node. Decision tree may include one or more internal nodes, defined as nodes connecting outputs of root nodes to inputs of terminal nodes. Computing device 408 may generate two or more decision trees, which may overlap; for instance, a root node of one tree may connect to and/or receive output from one or more terminal nodes of another tree, intermediate nodes of one tree may be shared with another tree, or the like.

With continued reference to FIG. 4, computing device 408 may build decision tree by following relational identification; for example, relational indication may specify that a first rule module receives an input from at least a second rule module and generates an output to at least a third rule module, and so forth, which may indicate to computing device 408 an in which such rule modules will be placed in decision tree. Building decision tree may include recursively performing mapping of panel outputs by one tree and/or subtree to root nodes of another tree and/or subtree. In this manner, computing device 408 may generate connections and/or combinations of one or more trees to one another to define overlaps and/or combinations into larger trees and/or combinations thereof. Such connections and/or combinations may be displayed by visual interface to user, for instance in first view, to enable viewing, editing, selection, and/or deletion by user; connections and/or combinations generated thereby may be highlighted, for instance using a different color, a label, and/or other form of emphasis aiding in identification by a user. In some embodiments, subtrees, previously constructed trees, and/or entire data structures may be represented and/or converted to rule modules, with graphical models representing them, and which may then be used in further iterations or steps of generation of decision tree and/or data structure. Alternatively or additionally subtrees, previously constructed trees, and/or entire data structures may be converted to APIs to interface with further iterations or steps of methods as described in this disclosure. As a further example, such subtrees, previously constructed trees, and/or entire data structures may become remote resources to which further iterations or steps of data structures and/or decision trees may transmit data and from which further iterations or steps of generation of data structure receive data, for instance as part of a decision in a given decision tree node.

Continuing to refer to FIG. 4, decision tree may incorporate one or more manually entered or otherwise provided decision criteria. Decision tree may incorporate one or more decision criteria using an application programmer interface (API). Decision tree may establish a link to a remote decision module, device, system, or the like. Decision tree may perform one or more database lookups and/or look-up table lookups. Decision tree may include at least a decision calculation module, which may be imported via an API, by incorporation of a program module in source code, executable, or other form, and/or linked to a given node by establishing a communication interface with one or more exterior processes, programs, systems, remote devices, or the like; for instance, where a user operating system has a previously existent calculation and/or decision engine configured to make a decision corresponding to a given node, for instance and without limitation using one or more elements of domain knowledge, by receiving an input and producing an output representing a decision, a node may be configured to provide data to the input and receive the output representing the decision, based upon which the node may perform its decision.

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

With continued reference to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example, input data may include subject data 116, ECG data 120, ECG feature, panel focuses 156a-n, panel outputs 136a-n, and the like. As a non-limiting illustrative example, output data may include ECG feature, panel focuses 156a-n, panel outputs 136a-n, pre-operative data structure 104, subject cohorts, and the like.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to subject cohorts. For example, and without limitation, subject cohorts may include cohorts of age, gender, lifestyle, smoking habits, alcohol consumption, weights, or the like.

With continued reference to FIG. 5, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

With continued reference to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

With continued reference to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include subject data 116, ECG data 120, ECG feature, panel focuses 156a-n, panel outputs 136a-n, and the like as described above as inputs, ECG feature, panel focuses 156a-n, panel outputs 136a-n, pre-operative data structure 104, subject cohorts, and the like as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

With continued reference to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

With continued reference to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

With continued reference to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

With continued reference to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

With continued reference to FIG. 5, redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

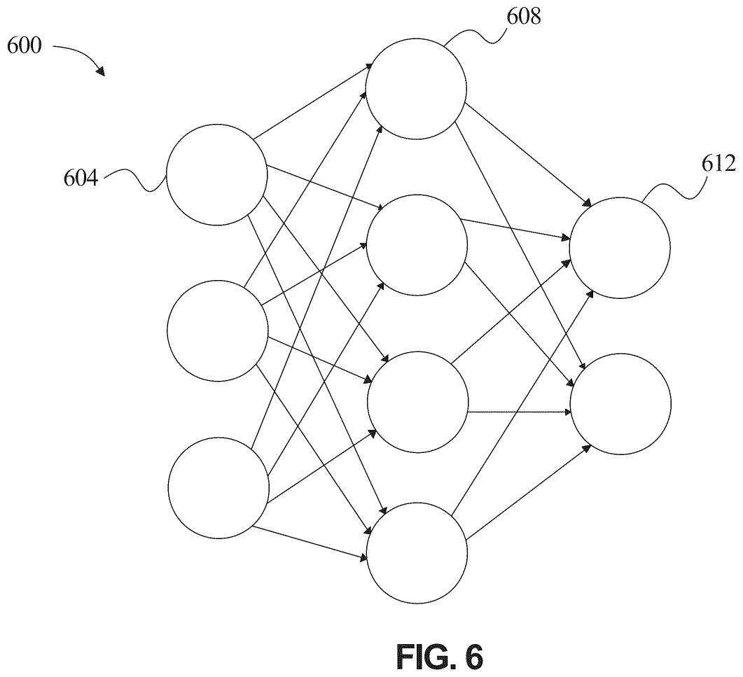

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

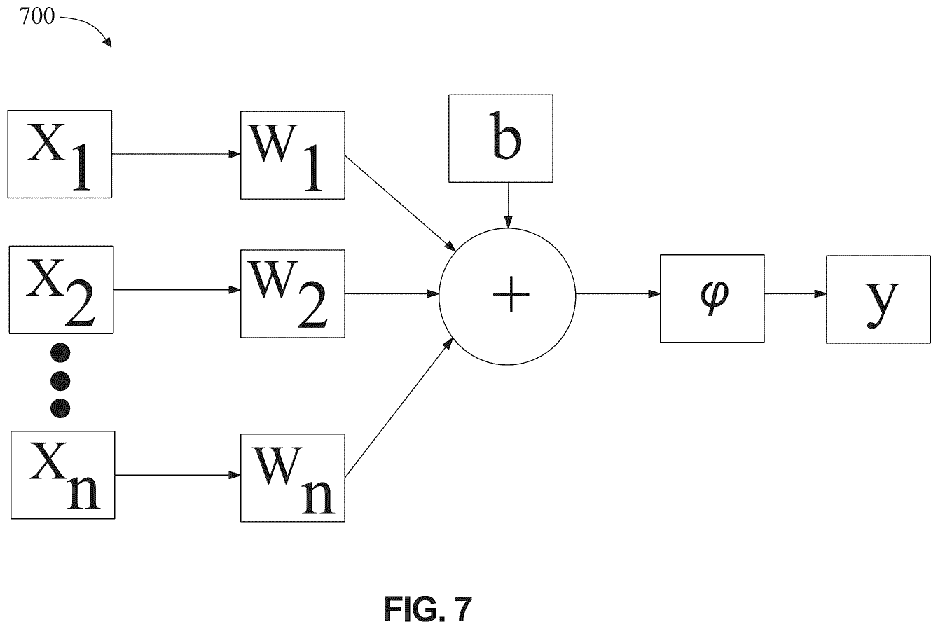

Referring now to FIG. 7, an exemplary embodiment of a node 700 of a neural network is illustrated. A node may include, without limitation, a plurality of inputs x¿ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tan h derivative function such as ƒ(x)=tan h²(x), a rectified linear unit function such as ƒ(x)=max(0,x), a “leaky” and/or “parametric” rectified linear unit function such as ƒ(x)=max(αx,x) for some α, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=α(1+tan h(\sqrt{2π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

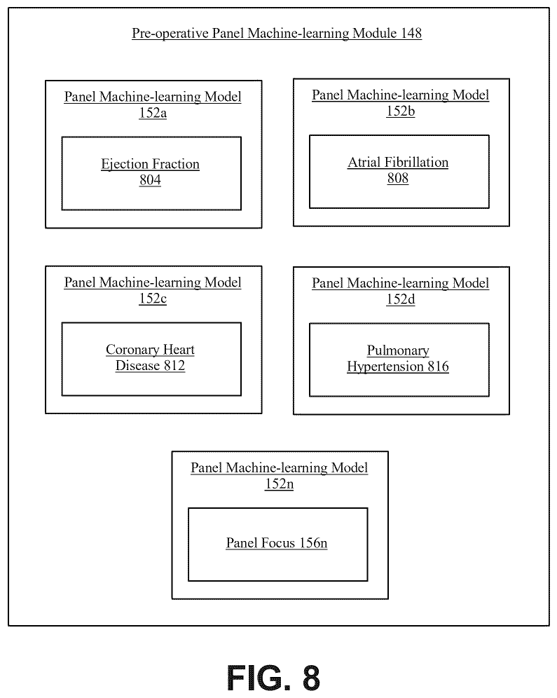

Referring now to FIG. 8, a block diagram of an exemplary pre-operative panel machine-learning module 148 is illustrated. Pre-operative panel machine-learning module 148 includes a plurality of panel machine-learning models 152a-n. As a non-limiting example, plurality of panel machine-learning models 152a-n may include a first panel machine-learning model 152a, second panel machine-learning model 152b, third panel machine-learning model 152c, fourth panel machine-learning model 152d, n number of panel machine-learning model 152n, or the like. Each of plurality of panel machine-learning models 152a-n is configured to generate one panel output 136a-n for one panel focus 156a-n as a function of subject data 116. In some embodiments, each plurality of panel machine-learning models 152a-n may include one panel focus 156a-n that is different compared to others. As a non-limiting example, panel focus 156a-n may include any medical conditions thereof. For example, and without limitation, panel focus 156a for panel machine-learning model 152a may include ejection fraction 804, panel focus 156b for panel machine-learning model 152b may include atrial fibrillation 808, panel focus 156c for panel machine-learning model 152c may include coronary heart disease 812, panel focus 156d for panel machine-learning model 152d may include pulmonary hypertension 816, panel machine-learning model 152n may include panel focus 156n, or the like. For example, and without limitation, panel focus 156a-n may further include complete blood count, coagulation studies, allergies, abnormal breathing, or the like. For example, and without limitation, panel focus 156a-n may further include a possibility of death before or after a surgery, necessity to get a surgery, alternative treatments, risks or complications of a surgery, type of anesthesia, cost of surgery, recovery time, or the like.

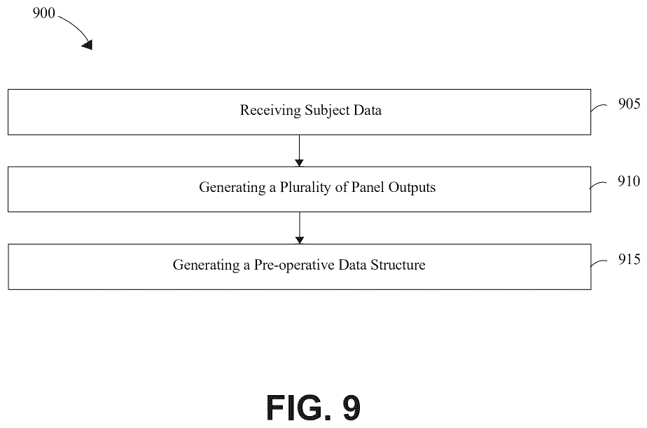

Referring now to FIG. 9, a flow diagram of an exemplary method 900 for generating a pre-operative data structure 104 using a pre-operative panel. Method 900 contains a step 905 of receiving, using at least a processor, subject data, wherein the subject data includes ECG data. This may be implemented as reference to FIGS. 1-8.

With continued reference to FIG. 9, method 900 contains a step 910 of generating, using at least a processor, a plurality of panel outputs as a function of subject data using a pre-operative panel machine-learning module, wherein the pre-operative panel machine-learning module includes a plurality of panel machine-learning models, wherein each of the plurality of panel machine-learning models is configured to generate one panel output for one panel focus as a function of the subject data, wherein generating the plurality of panel outputs includes generating a plurality of sets of panel training data, wherein the plurality of sets of panel training data includes correlations between exemplary subject data, exemplary panel focuses and exemplary panel outputs, training each of the plurality of panel machine-learning models using each of the plurality of sets of panel training data and generating the plurality of panel outputs using the plurality of trained panel machine-learning models. In some embodiments, generating the plurality of panel outputs may include determining, using the at least a processor, at least an ECG feature as a function of the ECG data and determining, using the at least a processor, the plurality of panel outputs as a function of the ECG feature. In some embodiments, determining the at least an ECG feature may further include generating, using the at least a processor, ECG feature training data, wherein the ECG feature training data may include correlations between exemplary ECG data and exemplary ECG features, training, using the at least a processor, an ECG feature machine-learning model using the ECG feature training data and determining, using the at least a processor, the at least an ECG feature using the trained ECG feature machine-learning model. In some embodiments, the plurality of panel machine-learning models may include a first panel machine-learning model including a first panel focus related to coronary heart disease, wherein the first panel machine-learning model may be configured to generate a first panel output related to the coronary heart disease as a function of the subject data. In some embodiments, the plurality of panel machine-learning models may include a second panel machine-learning model including a second panel focus related to pulmonary hypertension, wherein the second panel machine-learning model may be configured to generate a second panel output related to the pulmonary hypertension as a function of the subject data. In some embodiments, the plurality of panel machine-learning models may include a third panel machine-learning model including a third panel focus related to atrial fibrillation, wherein the third panel machine-learning model may be configured to generate a third panel output related to the atrial fibrillation as a function of the subject data. In some embodiments, the plurality of panel machine-learning models may include a fourth panel machine-learning model including a fourth panel focus related to ejection fraction, wherein the fourth panel machine-learning model may be configured to generate a fourth panel output related to the ejection fraction as a function of the subject data. In some embodiments, method 900 may further include generating, using the at least a processor, cohort training data, wherein the cohort training data may include correlations between exemplary subject data and exemplary subject cohorts, training, using the at least a processor, a cohort classifier using the cohort training data and classifying, using the at least a processor, the subject data to one or more subject cohorts using the trained cohort classifier. In some embodiments, method 900 may further include updating, using the at least a processor, the panel training data as a function of an output of the cohort classifier. In some embodiments, the plurality of panel outputs may include a pre-operative optimization output. These may be implemented as reference to FIGS. 1-8.

With continued reference to FIG. 9, method 900 contains a step 915 of generating, using at least a processor, a pre-operative data structure as a function of a plurality of panel outputs. This may be implemented as reference to FIGS. 1-8.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

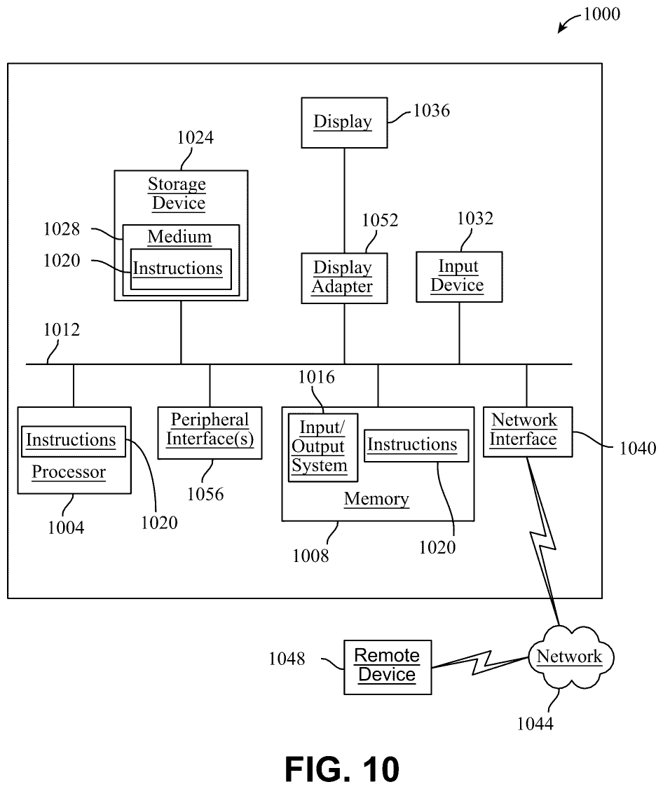

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and memory 1008 that communicate with each other, and with other components, via a bus 1012. Bus 1012 may include any of several types of bus structures including, but not limited to, memory bus, memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve apparatuses and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for generating a preoperative data structure using a pre-operative panel, the apparatus comprising:
at least a processor; and
a memory communicatively connected to the at least a processor, wherein the memory contains instructions configuring the at least a processor to:
receive subject data, wherein the subject data comprises electrocardiogram (ECG) data;
generate a plurality of panel outputs as a function of the subject data using a pre-operative panel machine-learning module;
wherein the pre-operative panel machine-learning module comprises a plurality of panel machine-learning models and a large language model (LLM), wherein each of the plurality of panel machine-learning models and the large language model are configured to generate one panel output for one panel focus as a function of the subject data;
wherein the large language model comprises a transformer architecture that employs self-attention and positional encoding, to receive the subject data as input, analyze the subject data, and generate an output;
wherein generating the plurality of panel outputs comprises:
generating a plurality of sets of panel training data, wherein the plurality of sets of panel training data comprises correlations between exemplary subject data, exemplary panel focuses and exemplary panel outputs, wherein generating the plurality of sets of panel training data comprises:
sanitizing the plurality of sets of panel training data using a dedicated hardware unit comprising circuitry configured to perform signal processing operations, wherein sanitizing the plurality of sets of panel training data comprises:
determining by the dedicated hardware unit that at least one training data entry of the plurality of sets of panel training data has a signal to noise ratio below a threshold value; and
removing the at least one training data entry from the plurality of sets of panel training data to create a sanitized plurality of sets of panel training data;
training each of the plurality of panel machine-learning models using each of the sanitized plurality of sets of panel training data; and
generating the plurality of panel outputs using the plurality of trained panel machine-learning models; and
generate a pre-operative data structure as a function of the plurality of panel outputs.

2. The apparatus of claim 1, wherein generating the plurality of panel outputs comprises:
determining at least an ECG feature as a function of the ECG data; and
determining the plurality of panel outputs as a function of the ECG feature.

3. The apparatus of claim 2, wherein determining the at least an ECG feature further comprises:
generating ECG feature training data, wherein the ECG feature training data comprises correlations between exemplary ECG data and exemplary ECG features;
training an ECG feature machine-learning model using the ECG feature training data; and
determining the at least an ECG feature using the trained ECG feature machine-learning model.

4. The apparatus of claim 1, wherein the plurality of panel machine-learning models comprises a first panel machine-learning model comprising a first panel focus related to coronary heart disease, wherein the first panel machine-learning model is configured to generate a first panel output related to the coronary heart disease as a function of the subject data.

5. The apparatus of claim 1, wherein the plurality of panel machine-learning models comprises a second panel machine-learning model comprising a second panel focus related to pulmonary hypertension, wherein the second panel machine-learning model is configured to generate a second panel output related to the pulmonary hypertension as a function of the subject data.

6. The apparatus of claim 1, wherein the plurality of panel machine-learning models comprises a third panel machine-learning model comprising a third panel focus related to atrial fibrillation, wherein the third panel machine-learning model is configured to generate a third panel output related to the atrial fibrillation as a function of the subject data.

7. The apparatus of claim 1, wherein the plurality of panel machine-learning models comprises a fourth panel machine-learning model comprising a fourth panel focus related to ejection fraction, wherein the fourth panel machine-learning model is configured to generate a fourth panel output related to the ejection fraction as a function of the subject data.

8. The apparatus of claim 1, wherein the memory contains instructions further configuring the at least a processor to:
generate cohort training data, wherein the cohort training data comprises correlations between exemplary subject data and exemplary subject cohorts;
train a cohort classifier using the cohort training data; and
classify the subject data to one or more subject cohorts using the trained cohort classifier.

9. The apparatus of claim 8, wherein the memory contains instructions further configuring the at least a processor to update the panel training data as a function of an output of the cohort classifier.

10. The apparatus of claim 1, wherein the plurality of panel outputs comprises a pre-operative optimization output.

11. A method for generating a preoperative data structure using a pre-operative panel, the method comprising:
receiving, using at least a processor, subject data, wherein the subject data comprises electrocardiogram (ECG) data;
generating, using the at least a processor, a plurality of panel outputs as a function of the subject data using a pre-operative panel machine-learning module;
wherein the pre-operative panel machine-learning module comprises a plurality of panel machine-learning models and a large language model (LLM), wherein each of the plurality of panel machine-learning models and the large language model are configured to generate one panel output for one panel focus as a function of the subject data;
wherein the large language model comprises a transformer architecture that employs self-attention and positional encoding, to receive the subject data as input, analyze the subject data, and generate an output;
wherein generating the plurality of panel outputs comprises:
generating a plurality of sets of panel training data, wherein the plurality of sets of panel training data comprises correlations between exemplary subject data, exemplary panel focuses and exemplary panel outputs, wherein generating the plurality of sets of panel training data comprises:
sanitizing the plurality of sets of panel training data using a dedicated hardware unit comprising circuitry configured to perform signal processing operations, wherein sanitizing the plurality of sets of panel training data comprises:
determining by the dedicated hardware unit that at least one training data entry of the plurality of sets of panel training data has a signal to noise ratio below a threshold value; and
removing the at least one training data entry from the plurality of sets of panel training data to create a sanitized plurality of sets of panel training data;
training each of the plurality of panel machine-learning models using each of the sanitized plurality of sets of panel training data; and
generating the plurality of panel outputs using the plurality of trained panel machine-learning models; and
generating, using the at least a processor, a pre-operative data structure as a function of the plurality of panel outputs.

12. The method of claim 11, wherein generating the plurality of panel outputs comprises:
determining, using the at least a processor, at least an ECG feature as a function of the ECG data; and
determining, using the at least a processor, the plurality of panel outputs as a function of the ECG feature.

13. The method of claim 12, wherein determining the at least an ECG feature further comprises:
generating, using the at least a processor, ECG feature training data, wherein the ECG feature training data comprises correlations between exemplary ECG data and exemplary ECG features;
training, using the at least a processor, an ECG feature machine-learning model using the ECG feature training data; and
determining, using the at least a processor, the at least an ECG feature using the trained ECG feature machine-learning model.

14. The method of claim 11, wherein the plurality of panel machine-learning models comprises a first panel machine-learning model comprising a first panel focus related to coronary heart disease, wherein the first panel machine-learning model is configured to generate a first panel output related to the coronary heart disease as a function of the subject data.

15. The method of claim 11, wherein the plurality of panel machine-learning models comprises a second panel machine-learning model comprising a second panel focus related to pulmonary hypertension, wherein the second panel machine-learning model is configured to generate a second panel output related to the pulmonary hypertension as a function of the subject data.

16. The method of claim 11, wherein the plurality of panel machine-learning models comprises a third panel machine-learning model comprising a third panel focus related to atrial fibrillation, wherein the third panel machine-learning model is configured to generate a third panel output related to the atrial fibrillation as a function of the subject data.

17. The method of claim 11, wherein the plurality of panel machine-learning models comprises a fourth panel machine-learning model comprising a fourth panel focus related to ejection fraction, wherein the fourth panel machine-learning model is configured to generate a fourth panel output related to the ejection fraction as a function of the subject data.

18. The method of claim 11, further comprising:
generating, using the at least a processor, cohort training data, wherein the cohort training data comprises correlations between exemplary subject data and exemplary subject cohorts;
training, using the at least a processor, a cohort classifier using the cohort training data; and
classifying, using the at least a processor, the subject data to one or more subject cohorts using the trained cohort classifier.

19. The method of claim 18, further comprising:
updating, using the at least a processor, the panel training data as a function of an output of the cohort classifier.

20. The method of claim 11, wherein the plurality of panel outputs comprises a pre-operative optimization output.
